(19) United States
(12) Patent Application Publication
(10) Pub. No.: US 2003/0129649 A1
(43) Pub. Date: Jul. 10, 2003

(54) Conformational assays to detect binding to G protein-coupled receptors

(76) Inventors: Brian K. Kobilka, Palo Alto, CA (US); Pejman Ghanouni, Menlo Park, CA (US); Tae Weon Lee, Palo Alto, CA (US)

Correspondence Address: BOZICEVIC, FIELD & FRANCIS LLP, 200 MIDDLEFIELD RD, SUITE 200, MENLO PARK, CA, 94025, US

(21) Appl. No.: 09/935,061

(22) Filed: Aug. 21, 2001

Related U.S. Application Data
(60) Provisional application No. 60/286,250, filed on Apr. 24, 2001.

(51) Int. Cl. G01N 33/53; G01N 33/567; C12M 1/34

(52) U.S. Cl. 435/7.1; 435/7.2; 435/287.2

(57) ABSTRACT

The present invention provides methods and compositions for detection of compounds that have activity in modulating G protein-coupled receptor (GPCR) activity, e.g., agonists, and antagonists. The detection method is based upon detection of a conformational change in a GPCR upon interaction with a ligand. Conformational change of the GPCR upon ligand interaction can be accomplished by modifying the GPCR to have a bound detectable label so that ligand interaction results in a conformational change in the GPCR that is detected by a change in detectable signal from the detectable label. Conformational change of the GPCR upon ligand interaction can also be detected by detecting a change in the accessibility of a protease cleavage site to protease cleavage, where the protease cleavage site is naturally-occurring in the GPCR or introduced into the GPCR. The conformational assays of the invention provide for high-throughput screening,

### CROSS-REFERENCE TO RELATED APPLICATION

This application claims the benefit of earlier-filed U.S. provisional application serial No. 60/286,250, filed Apr. 24, 2001, which application is incorporated herein by reference in its entirety.

### GOVERNMENT RIGHTS

The United States Government may have certain rights in this application pursuant to Grant 5RO1 NS28471.

### FIELD OF THE INVENTION

This invention relates to methods of detecting G-protein coupled receptor (GPCR) activity, and methods of screening for GPCR ligands and other compounds that interact with components of the GPCR regulatory process.

### BACKGROUND OF THE INVENTION

Despite diverse physiologic roles, the majority of G protein coupled receptors (GPCRs) are thought to share a common activation mechanism. Briefly, agonists induce conformational changes in receptors, which then stimulate heterotrimeric GTP-binding proteins (G proteins). Activated G proteins influence cellular physiology by modulating specific effector enzymes and ion channels involved in cardiovascular, neural, endocrine, and sensory signaling systems (see, e.g., Strader et al., Annu Rev Biochem 63:101-32 (1994)).

The actions of many extracellular signals are mediated by the interaction of guanine nucleotide-binding regulatory proteins (G proteins) and G-protein coupled receptors (GPCRs). Individual GPCRs activate particular signal transduction pathways through binding to G proteins, which in turn transduce a signal to the cell to elicit a response from the cell. GPCRs are known to respond to numerous extracellular signals, including neurotransmitters, drugs, hormones, odorants and light. The family of GPCRs has been estimated to include several thousands members, filly more than 1.5% of all the proteins encoded in the human genome. The GPCR family members play roles in regulation of biological phenomena involving virtually every cell in the body. The sequencing of the human genome has led to identification of numerous GPCRs; although the ligands and functions of many of these GPCRs are known, a significant portion of these identified receptors are without known ligands. These latter GPCRs, known as “orphan receptors”, also generally have unknown physiological roles.

Many available therapeutic drugs in use today target GPCRs, as they mediate vital physiological responses, including vasodilation, heart rate, bronchodilation, endocrine secretion, and gut peristalsis. See, eg., Lefkowitz et al., Ann. Rev. Biochem. 52:159 (1983); Gilman, A. G. (1987) Annu. Rev. Biochem 56: 615-649; Hamm, H. E. (1998) JBC 273: 669-672; Ji, T. H. (1998) JBC 273:17229-17302; Kanakin, T. (1996) Pharmacological Review, 48:413-463; Gudermann T. and Schultz, G. (1997), Annu. Rev. Neurosci., 20: 399-427. In fact, it has been estimated that more than 50% of the drugs in use clinically in humans at the present time are directed at GPCRs, including the adrenergic receptors (ARs). For example ligands to beta ARs are used in the treatment of anaphylaxis, shock, hypertension, hypotension, asthma and other conditions.

Since GPCRs and G protein signaling pathways are critical targets for therapeutics, there is a need in the art for fast, effective and reproducible methods for identifying agonists, antagonists and inverse agonists that modulate G protein signaling, and in particular compounds that regulate this signaling through a GPCR. In general, three different approaches to identify compounds that interact with GPCRs have been described. A first approach for identification of agents that activate GPCRs is based on the ability of the compound to bind to a GPCR, e.g., as in a competitive binding assay. Binding assays measure the ability of a compound to displace the binding of a known ligand to the receptor. They are limited by the availability of such ligands and are therefore not useful for orphan GPCRs. This approach generally requires that the natural ligand of the GPCR be known, particularly where the assay is based upon competitive binding. This approach is thus not useful with orphan GPCRs.

A second approach is to screen candidate agents for the ability to activate GPCR function, e.g., a functional assay. Signaling assays measure the ability of ligands to activate components of a signal transduction cascade, such as G protein or second messenger activation (Tota et al. (1990) Mol Pharmacol 37(6), 996-1004; Selley, et al. (1997) Mol Pharmacol 51(1), 87-96; Krumins, et al. (1997) Mol Pharmacol 52(1), 144-54; 4. Perez, et al. (1996) Mol Pharmacol 49(1), 112-22). These assays are best suited for detecting agonists and the effectiveness of the assay is somewhat dependent on the receptor's G protein coupling specificity. In the case of orphan GPCRs, this coupling specificity is not known.

A third approach involves detection of conformational changes. Several biophysical studies on the β₂AR and rhodopsin have demonstrated conformational changes in TM6 or the attached intracellular loop 3 (IC3) region upon ligand activation (Sheikh, et al. (1996) Nature 383(6598), 347-50; Altenbach, et al. (1996) Biochemistry 35(38), 12470-8; Farrens, et al. (1996) Science 274(5288), 768-70; Gether, et al. (1997) Embo J 16(22), 6737-47). However, the techniques in these studies require labeling of multiple sites in the receptor and/or are not amenable to high throughput screening (e.g., the assays do not provide a large enough difference in detectable signal to make the assay useful in high throughput screening). Other conventional techniques focus upon the use of surface plasmon resonance techniques, which are tedious, time consuming, and not easily adapted to high-throughput screening.

There is a need in the field for assays for detection of candidate agents that modulate activity of GPCRs, and which can be readily adapted to high-throughput screening of candidate agents. The present invention addresses this need.

### SUMMARY OF THE INVENTION

The present invention provides methods and compositions for detection of compounds that have activity in modulating G protein-coupled receptor (GPCR) activity, e.g., agonists, and antagonists. The detection method is based upon detection of a conformational change in a GPCR upon interaction with a ligand. Conformational change of the GPCR upon ligand interaction can be accomplished by modifying the GPCR to have a bound detectable label so that ligand interaction results in a conformational change in the GPCR that is detected by a change in detectable signal from the detectable label. Conformational change of the GPCR upon ligand interaction can also be detected by detecting a change in the accessibility of a protease cleavage site to protease cleavage, where the protease cleavage site is naturally-occurring in the GPCR or introduced into the GPCR. The conformational assays of the invention provide for high-throughput screening.

In one aspect, the invention provides methods for identifying candidate agents that modulate activity of a GPCR by detection of a conformational change upon interaction with the candidate agent. Detection of a conformational change indicates the candidate agent has activity in modulating GPCR activity. In one embodiment, the conformational change is detected by a change in signal of a detectable label attached to the GPCR being tested. In another embodiment, the conformational change is detected by a change in the accessibility of a protease cleavage site in the GPCR or modified GPCR to cleavage by the protease.

In another embodiment, the invention provides an apparatus for detecting G protein coupled receptor (GPCR) activity that comprises 1) a plurality of GPCRs, each GPCR or a portion thereof inserted into a membrane; and 2) an immobilization phase. Each GPCR is identifiably placed in the apparatus such that its particular activity in response to an agent (e.g. a ligand) can be determined relative to the activity of the other GPCRs. The immobilization phase can be any appropriate solid or semi-solid phase, e.g., an assay plate (e.g., a microtiter plate comprising well) or a flat surface (e.g., a glass slide). The surface of the immobilization phase can be modified to allow for specific and/or oriented interaction of the receptor with the surface.

In another embodiment, the present invention provides a method of detecting G protein coupled receptor (GPCR) activity for a plurality of GPCRs by contacting an apparatus with an agent, where the apparatus comprises a plurality of GPCRs inserted into a plurality of membranes (e.g., an enriched plasma membrane fraction) or a portion thereof, and detecting activity of each receptor in response to the agent. Where receptor activity is detected by a change in signal generated by a detectable label, the signal can be detected by photochemical (e.g., fluorescent), biochemical or other means, and can be detected at discrete time points or as a function over time. Alternatively, the detectable signal is provided by detection of a change in the accessibility of a protease cleavage site present in the GPCR to protease cleavage. Protease cleavage can be detected by detection of protease cleavage products, e.g., by detection of a newly formed internal C-terminus that is produced by protease cleavage, or by detection of the presence or absence of one or more cleavage products.

The methods of the invention can be performed with GPCRs of known function, for example to identify agents that increase or decrease (e.g., modulate) GPCR activity involved in a certain biological process, or with an “orphan” GPCR, for example to aid in determining its function based on modulation by a known ligand.

In another embodiment, the present invention provides a method of identifying an agent that modulates a GPCR by contacting an apparatus with an agent, where the apparatus comprises a plurality of GPCRs inserted into a whole cell membrane or a portion thereof, and detecting activity of each receptor in response to the agent by detection of a conformational change in the GPCR as described above. A change in activity of the GPCR is indicative of an agent that modulates GPCR activity, and the type of activity change can allow classification of the molecule as an agonist, an antagonist, or an inverse agonist. The change in activity can be determined by comparing activity of the GPCR with and without the agent, and/or by comparing levels of GPCR activity in the presence of the agent with a standard.

One object of the present invention is to provide rapid and sensitive bioassays for evaluating new agonists, antagonists and/or inverse agonists for GPCRs.

Another object of the present invention is to identify ligands for GPCRs.

Yet another object of the present invention is to identify GPCRs involved in different biological processes, including disease.

Yet another object of the invention is to identify the presence of a particular ligand in a sample, e.g., the presence of a drug such as an opioid.

An advantage of one embodiment of the invention (protease) is that the assays can be performed using membranes, which increases both the ease of performing the assay and the efficacy of the assay.

Another advantage is that assays of the invention allow high throughput screening of GPCR activity.

Yet another advantage of the invention is that it allows for determination of the affinity of a ligand for a GPCR.

Still another advantage of the invention is that, when provided in an array format, the invention can provide for determination of ligand specificity with a specific GPCR on the array.

These and other objects, advantages, and features of the invention will become apparent to those persons skilled in the art upon reading the details of the apparatus and assays as more fully described below.

### BRIEF DESCRIPTION OF THE DRAWINGS

FIGS. 1A-1C are schematic diagrams of the secondary structure of β₂AR illustrating the fluorescein maleimide (FM) labeling site at Cys265.

FIG. 1A illustrates the position of the 13 cysteines (C in a circle) in the β₂AR, yet only Cys265 is labeled with the relatively large, polar fluorophore FM under the conditions described in the Methods below. Cysteine residues are indicated by circles; asparatic acid residues by D in a circle; phenylalanine by F in a circle; and serine by S in a circle. Cys106, Cys184, Cys190, and Cys191 have been shown to be disulfide bonded and Cys341 is palmitoylated. Cys378 and Cys406 in the carboxyl terminus form a disulfide bond during purification. Labeling specificity was confirmed by peptide mapping and mutagenesis of potential reactive cysteines (data not shown). The sites of peptide cleavage by Factor Xa (line) and cyanogen bromide (black dots) are shown.

FIG. 1B is a schematic of transmembrane helices 5 and 6 and the connecting intracellular loop 3 (IC3). The location of the fluorescein maleimide (F) site is highlighted. Fluorescence quenchers (squares) localized to either the aqueous milieu, the micellar environment, or to the base of TM5 (oxyl-NHS bound to Lys224, red square) were used to monitor conformational changes around Cys265.

In FIG. 1C, cylinders representing the seven transmembrane helices of the β₂AR as viewed from the cytoplasmic side of the membrane, arranged according to the crystal structure of rhodopsin in the inactive state. In the inactive receptor, FM on Cys265 is predicted to point toward the cytoplasmic extensions of transmembranes 3, 5, and 6. Also shown is the predicted position of the quencher oxyl-NHS on Lys224 (square).

FIGS. 2A-2B illustrate the effect of agonists and partial agonists on fluorescence intensity of FM-β₂AR.

In FIG. 2A, the change in intensity of FM-b2AR in response to the addition of the full agonist (−)-isoproterenol (ISO) and the strong partial agonist epinephrine (EPI) was reversed by the neutral antagonist (−)-alprenolol (ALP). FIG. 2B illustrates the agonist and partial agonist effects on the intensity of FM-β2AR compared with an assay of biological efficacy (GTPγS binding).

FIGS. 3A-3B illustrate the response of FM-β₂AR to agonist in the presence of KI or Oxyl-NHS. FIG. 3A is a Stem-Volmer plots of KI quenching of FM-labeled β₂AR. FIG. 3B shows the effect of quenchers KI and Oxyl-NHS on the magnitude of the ISO-induced decrease in fluorescence.

FIGS. 4A-4D provide a comparison of effects of quenchers localized to the micelle on the response of FM-β_{2A}R to (−)-isoproterenol.

FIG. 4A is a schematic depicting the structure of CAT-16 and 5-doxyl stearate (5-DOX), as well as the putative location of these quenching groups in the micelle. The quenching group on 5-DOX is located within the hydrophobic core of the micelle.

FIG. 4B is a Stern-Volmer plot depicting the extent of quenching of FM-β2AR by increasing concentrations of CAT-16 or 5-DOX.

FIG. 4C illustrates the differing effects of CAT-16 and 5-DOX on agonist-induced fluorescence change of FM-β2AR. The extent of response to (−)-isoproterenol is presented as a % control ISO response, calculated as in FIG. 3.

FIG. 4D is an example of the experiments used to generate the ratios in FIG. 4C.

FIGS. 5A and 5B are schematics showing agonist-induced conformational changes in TM6. The model represents TM 3, 5, and 6 as viewed from the cytoplasmic surface of the receptor arranged according to the crystal structure of rhodopsin. FM on Cys265 is indicated by the circle; oxyl-NHS on Lys224 is indicated by the square. The results from quenching experiments can best be explained by either a clockwise rotation of TM6 (FIG. 5A) and/or tilting of TM6 (FIG. 5B) toward TM5 during agonist-induced activation of the receptor.

FIG. 6A is a schematic diagram of the secondary structure of β2 AR illustrating the fluorescein maleimide (FM) labeling site at Cys265. Amino acids in dark circles have been shown to be important for agonist binding.

FIG. 6B is a graph showing the effect of the full agonist (−)-isoproterenol (ISO) on fluorescence intensity of FM-β2AR. Purified, detergent-solubilized β2-AR was labeled with FM at Cys265 and examined by fluorescence spectroscopy. Change in intensity of FM-b2 AR in response to the addition of ISO followed by the reversal by the neutral antagonist (−)-alprenolol (ALP).

FIG. 7 is a graph showing the effect of drugs on fluorescence lifetime distributions of FM-β2 AR. Fluorescence lifetimes were determined by phase modulation and lifetime distributions of FM-β2 AR were calculated in the absence of ligand, with the neutral antagonist ALP, or in the presence of the full agonist ISO. The mean lifetime and the full width at half maximum for the distributions are: No Ligand τ=4.21±0.01 nsec, FWHM=1.1±0.1, χ²=2.8; ALP: τ=4.21±0.01 nsec, FWHM=0.7±0.2, χ²=2.9; ISO: σ_{LONG}=4.36 ±0.08 nsec, FWHM_{LONG}=0.5±1.1, χ_{SHORT}=0.76±0.33 nsec, FWHM_{SHORT}=1.7±1.2, χ²=3.2.

FIGS. 8A and 8B are graphs showing the comparison of the effects of full and partial agonists on the fluorescence lifetime distributions of FM-β2 AR. In FIG. 8A the effect of the full agonist ISO and partial agonists SAL and DOB on the lifetime distributions of FM-β2 AR are compared. FIG. 8B provides an expanded view of the short lifetime distributions shown in FIG. 8A. The mean lifetime and the full width at half maximum for the new distributions are: SAL: τ_{LONG}=4.37±0.04 nsec, FWHM_{LONG}=0.7±0.3, τ_{SHORT}=1.93±0.24 nsec, FWHM_{SHORT}=0.7±0.3, χ²=2.1; DOB: τ_{LONG}=4.38±0.01 nsec, FWHM_{LONG}=0.4±0.4, τ_{SHORT}=1.78±0.01, FWHM_{SHORT}=0.9±0.6, χ²=2.0.

FIGS. 9A-9B are diagrams of the two-state model of GPCR activation. In FIG. 9A, R is the inactive conformation and R* is the active conformation capable of activating the G protein. The equilibrium between R and R* is influenced differently by agonists (ISO) and partial agonists (DOB). The width of the arrows reflects the rate constant. FIG. 9B is a diagram of a multistate model of GPCR activation. The agonist ISO and the partial agonist DOB both induce an intermediate state R′, as well as distinct G protein activating conformations R* and R^{X}, respectively. The neutral antagonist ALP induces a conformation R^{o} that is functionally equivalent to R at activating the G protein Gs, but can be distinguished from R by susceptibility to digestion by proteases.

FIGS. 10A-10B show the effect of agonists and antagonists on susceptibility of GPCR to trypsin cleavage. FIG. 10A shows that fluorescence of FM-β2-AR increases upon exposure to the protease trypsin. FIG. 10B shows the change in fluorescence when the GPCR is pretreated with H2) (control), ISO, DOB, or ALP.

FIG. 11 is schematics showing a GPCR having a protease cleavage site positioned so that ligand binding results in a conformational change that renders the protease cleavage site accessible to protease cleavage.

FIG. 12 is a schematic showing the amino acid sequence of β₂-adrenergic receptor and modifications that can be made within the second intracellular loop or within the third intracellular loop to insert a protease cleavage site (exemplified by tobacco etch virus (TEV)) that can serve as a conformationally sensitive probe for ligand binding.

FIG. 13 is a schematic showing the DNA and amino acid sequence of the of β₂-adrenergic receptor.

FIG. 14 is a schematic showing the DNA and amino acid sequence of a β₂-adrenergic receptor modified to contain a TEV protease cleavage site in the second intracellular loop.

FIG. 15 is a schematic showing the DNA and amino acid sequence of a β₂-adrenergic receptor modified to contain a TEV protease cleavage site in the third intracellular loop.

FIG. 16 is a schematic showing the amino acid sequence of μ-opioid receptor and modifications that can be made within the second intracellular loop or within the third intracellular loop to insert a protease cleavage site (exemplified by tobacco etch virus (TEV)) that can serve as a conformationally sensitive probe for ligand binding.

FIG. 17 is a schematic showing the DNA and amino acid sequence of a opioid receptor.

FIG. 18 is a schematic showing the DNA and amino acid sequence of a opioid receptor modified to contain a TEV protease cleavage site in the second intracellular loop.

FIG. 19 is a schematic showing the DNA and amino acid sequence of a opioid receptor modified to contain a TEV protease cleavage site in the third intracellular loop.

### DETAILED DESCRIPTION OF INVENTION

Before the present assays and methods are described, it is to be understood that this invention is not limited to particular protocols and/or embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a GPCR” includes a plurality of such GPCRs and reference to “the ligand” includes reference to one or more ligand and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DEFINITIONS

The term “agonist” as used herein refers to a molecule or substance that binds to or otherwise interacts with a receptor or enzyme to increase activity of that receptor or enzyme. Agonist as used herein encompasses both full agonists and partial agonists.

The term “antagonist” as used herein refers to a molecule that binds to or otherwise interacts with a receptor to block (e.g., inhibit) the activation of that receptor or enzyme by an agonist.

The term “inverse agonist” as used herein refers to a molecule that binds to or otherwise interacts with a receptor to inhibit the basal activation of that receptor or enzyme.

The term “ligand” as used herein refers to a naturally occurring or synthetic compound that binds to a protein receptor. Upon binding to a receptor, ligands generally lead to the modulation of activity of the receptor. The term is intended to encompass naturally occurring compounds, synthetic compounds and/or recombinantly produced compounds. As used herein, this term can encompass agonists, antagonists, and inverse agonists.

The term “receptor” as used herein refers to a protein normally found on the surface of a cell which, when activated, leads to a signaling cascade in a cell.

The term “functional interaction” as used herein refers to an interaction between a receptor and ligand that results in modulation of a cellular response. These may include changes in membrane potential, secretion, action potential generation, activation of enzymatic pathways and long term structural changes in cellular architecture or function.

The term “G protein subunit” as used herein can refer to any of the three subunits, α, β or γ, that form the heterotrimeric G protein. The term also refers to a subunit of any class of G protein, e.g. Gₛ, Gᵢ/Gₒ, G_{q} and G_{z}. In addition, recitation of a specific subunit (e.g., Gα) is intended to encompass that subunit in each of the different classes, unless the class of G protein is specifically otherwise specified.

The terms “G protein coupled receptors” and “GPCRs” as used interchangeably herein include all subtypes of the opioid, muscarinic, dopamine, adrenergic, adenosine, rhodopsin, angiotensin, serotonin, thyrotropin, gonadotropin, substance-K, substance-P and substance-R receptors, melanocortin, metabotropic glutamate, or any other GPCR known to couple via G proteins. This term also includes orphan receptors that are known to couple to G proteins, but for which no specific ligand is known.

The term “conformationally sensitive detectable probe” as used herein refers to a moiety on a naturally occurring or modified GPCR that provides a change in a detectable signal upon interaction of the GPCR with a ligand, particularly with a ligand having agonist activity (e.g., activity as a full or partial agonist). One exemplary conformationally sensitive detectable probe is a detectable label (e.g., a fluorescent moiety) that is attached to an amino acid residue within the third intracellular loop of a GPCR (e.g., an amino acid residue corresponding to Cys265 of β2-AR), so that interaction of the GPCR with an agonist results in a change in the detectable signal of the detectable label (e.g., a decrease in signal due to agonist binding). Another exemplary conformationally sensitive detectable probe is a protease cleavage site (either naturally occurring or introduced using recombinant techniques) within the third intracellular loop of the GPCR, so that the protease cleavage site becomes more or less accessible following interaction with an agonist.

The terms “epitope tagged protein” and the like are used interchangeably herein to mean an artificially constructed proteins having one or more heterologous epitope domain(s).

The term “biological system” as used herein refers to any system in which the molecular responses to the activation of G proteins, e.g., activation through GPCRs, can be measured. The biological systems may be in vitro (e.g., membrane preparations or cell culture).

By “immobilization phase” is meant a matrix to which the membrane preparation can attach. The immobilization phase can be of any suitable form including solid, semi-solid, and the like. Usually, the immobilization phase comprises the well of an assay plate but the invention is by no means limited to this embodiment. For example, the immobilization phase can comprise a discontinuous immobilization phase of discrete particles, or it may comprise a flat surface. The immobilization phase can be formed from a number of different materials, e.g., polysaccharides (e.g. agarose), polyacrylamides, polystyrene, polyvinyl alcohol, silicones and glasses. The surface of the immobilization phase can be modified to allow for specific and/or oriented interaction of the receptor with the surface.

By “membrane” is meant plasma membrane or fragment from a eukaryotic cell (e.g., insect) or artificial membrane (e.g., detergent micelle).

By “well” is meant a recess or holding space in which an aqueous sample can be placed. The well is provided in an “assay plate” which is formed from a material (e.g. polystyrene) which optimizes adherence of cells (having the receptor or receptor construct) or membrane preparations thereto. The individual wells of the assay plate can have any suitable shape, including but not limited to a round bottom well and a flat bottom well. In a particular embodiment of the invention, the assay plate comprises between about 30 to 200 individual wells, usually 96 wells, and is designed to allow for automation of the assay.

The abbreviations used herein include:

GPCR for G protein-coupled receptor;

β2 AR (or b2AR or beta2AR) for β2 adrenoceptor;

FM for fluorescein maleimide;

Gα, for an a subunit of a G-protein

Gₛα, for an α subunit of the stimulatory G-protein;

AC for adenylyl cyclase;

(³H)DHA for (³H)dihydroalprenol;

GTPγS for guanosine 5′-O-(3-thiotriphosphate);

ISO for (−)isoproterenol;

DOB for dobutamine;

ALP for (−) alprenolol; and

ICI for ICI-118,551.

Overview

The present invention is based on the discovery that conformationally sensitive probes can be used to detect interactions between GPCRs and ligands by direct detection of ligand-induced conformational changes in the receptor protein.

Monitoring of ligand-induced conformational change is accomplished by modifying the receptor protein with a conformationally sensitive probe at a specific site on the protein. This modification is accomplished by generating modified receptors using site-directed mutagenesis. The modifications are limited to cytoplasmic domains of the receptor and therefore do not alter sequences involved in ligand binding.

There are several types of conformational probes. This invention encompasses the use of fluorescent molecules and site-specific proteases as such conformational probes, as well as electron paramagnetic resonance (EPR) probes and nuclear magnetic resonance (NMR) probes. Using conformationally sensitive probes, receptor-ligand interactions can be monitored using, for example, a fluorescence-based assay. In the case where receptor protein is labeled directly with the fluorescent probe, the interaction assay can be performed with purified, detergent solubilized receptor protein. In the case where the receptor protein is modified with a site-specific protease, the interaction assay can be performed on purified receptor protein or on receptor-enriched membrane fragments. All embodiments of the invention allow the generation of arrays consisting of different G protein coupled receptors such that GPCR-ligand interactions could be assessed in multiple receptors simultaneously.

GPCRs

Exemplary GPCRs that can be used in the screening assays of the invention include, but are not necessarily limited adrenoceptors, opioid receptors, and the like. Further exemplary GPCRs that can be used in the present invention are listed in the table below. The GPCRs are classified according to the type of ligand they naturally bind.

Table of Exemplary GPCRs

| | | Other Receptors |
|---|---|---|
| Peptide ligands | | |
| Angiotensin receptors | Releasing hormone | |
| | receptors (LHRH, GHRH) | |
| Bombesin receptors | Somatostatin receptors | |
| Bradykinin receptors | Tachykinin receptors | |
| Calcitonin, parathyroid | Thrombin/protease | |
| hormone, secretin | receptors | |
| receptors | | |
| Chemokine receptors | Vasopressin/oxytocin | |
| | receptors | |
| Chemotactic peptide | Glycoprotein hormones | Odorant/olfactory |
| receptors (fMLP) | receptors (TSH, FSH, LH) | and gustatory |
| | | receptors |
| C5A receptor | Melanocortins receptors | Opsins |
| Cholecystokinin/ | Neuropeptide Y receptors | Viral receptors |
| gastrin receptors | | |
| Corticotropin (ACTH) | Neurotensin receptors | Orphan receptors |
| receptor | | |
| Endothelin receptors | Opioid peptides receptors | |
| | (mu, delta, kappa & opioid | |
| | like) | |

Natural small molecule ligands

| | | |
|---|---|---|
| Acetylcholine | Dopamine receptors | Prostanoids and |
| (muscarinic) receptors | | PAF receptors |
| Adenosine and adenine | Histamine receptors | Serotonin receptors |
| nucleotide receptors | | |
| Adrenergic receptors | Cannabinoids receptors | Metabotropic |
| | | glutamate and |
| | | calcium receptors |

The GPCRs that are involved in biological responses, both normal responses (e.g., taste, smell, etc.) and pathological responses (e.g., the biological response to a disease-related protein) can be determined using assays and apparatus of the invention. An assay using an array of membranes or proteins, each sample of the array having a particular GPCR of interest, can be exposed to the stimulus (e.g., the odor, flavor compound, disease related complex, and the like), and the activity of each sample of the array can be determined. This can identify multiple receptors in a high-throughput manner that are involved in the transduction of signals in response to the stimulus.

The high-throughput assays of the invention can be especially useful in determining the spectrum of GPCRs, e.g., olfactory receptors, that are activated or inverse agonized by a specific substance or mixture of substances. For example, a liquid can be contacted with an array of membrane preparations each having a particular GPCR of interest, and the GPCRs activated or suppressed can be identified by detection of a conformational change in the GPCR. This can classify the liquid (e.g., a perfume or a beverage) for a specific market or to identify compounds important in creating the liquid.

In another example, an assay using the apparatus of the invention can be used to identify the ligands that bind to and modulate GPCRs of unknown activity, e.g., orphan receptors. Identification of ligands that modulate specific receptors can lead to a better understanding of the functional role of that particular receptor.

Other uses are also envisioned, as will be apparent to one skilled in the art upon reading the present disclosure.

Assays of the Present Invention

Methods for detecting or identifying G protein activation through GPCRs are important for numerous applications in medicine and biology. The present invention provides methods including: (1) methods for rapidly and reproducibly screening for new drugs affecting selected GPCRs, (2) methods for identifying the native ligand for orphan GPCRs, and (3) methods for detecting the presence of known chemicals that associate with GPCRs in a sample, e.g., drugs that activate GPCRs. The basic assays described herein and variations thereof can also be used in other applications, as will be apparent to those skilled in the art upon reading the present application.

A significant advantage of the assays of the invention is that they can directly detect interaction of a compound with a GPCR either qualitatively or quantitatively, and thus are particularly amenable to high-throughput screening of large numbers of GPCRs. For example, the assay can be conducted using two or more different GPCRs, where different GPCRs can be different due to differences in naturally-occurring or artificially-induced amino acids sequences (e.g., a native (i.e., naturally-occurring) and mutated version of a βAR are different GPCRs, a native βAR and a native opioid receptor are different GPCRs, etc.).

The assay can be conducted using a plurality of different GPCRs (e.g., three or more, five or more, ten or more, twenty or more, and the like). The different GPCRs can be provided in membranes or micelles, or can be provided in the membrane or micelle, where induction of activity of the GPCRs can be detected using different detectable labels. Detection of activity of compounds on different GPCRs can be accomplished by differential labeling of the GPCRs (e.g., particularly where two or more GPCRs are provided in the same membrane). In general, a plurality of GPCRs can be screened by distinguishing the different GPCRs based on their location on an array (e.g., each GPCR is positioned on an immobilization phase at a known coordinate, so that detection of a change in detectable label at that coordinate (e.g., detection of a change in fluorescent signal at that coordinate) can be associated with activity of the compound on the GPCR at that same coordinate).

The GPCRs screened can represent a diverse collection of GPCRs, or can represent a collection of GPCRs having a role in a biological phenomenon of interest. This can be useful, for example, in determining the receptors activated by a particular drug or receptors that are activated upon exposure to a particular stimulus, such as an odor or taste (e.g., activation of olfactory GPCRs)

Production of GPCRs (for modification and labeling) or modified GPCRs (by insertion of a protease cleavage site) can be any suitable host cell (e.g., mammalian, yeast, insect, or bacterial). In one embodiment of particular interest, the host cells are insect cells. Methods for expression of recombinant GPCRs, as well as methods for isolation of such recombinant GPCRs and methods of production of membranes containing GPCRs, are well known in the art (see, e.g., Kobilka Anal. Biochem. 231(1):269-71 (1995); Gether et al. J Biol. Chem. 270(47):28268-75 (1995)).

Candidate Agents

Identification of compounds that modulate GPCR activity can be accomplished using any of a variety of drug screening techniques as described in more detail below. Of particular interest is the identification of agents that have activity in affecting GPCR function. Such agents are candidates for development of treatments for, conditions associated at least in part with GPCR activity. Of particular interest are screening assays for agents that have a low toxicity for human cells. The term “agent” as used herein describes any molecule, e.g. protein or pharmaceutical, with the capability of altering (i.e., eliciting or inhibiting). Generally a plurality of assay mixtures are run in parallel with different agent concentrations to obtain a differential response to the various concentrations. Typically, one of these concentrations serves as a negative control, i.e. at zero concentration or below the level of detection.

Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including, but not limited to: peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts (including extracts from human tissue to identify endogenous factors affecting GPCRs) are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Screening Assays

In general, the assays of the invention involve detection of a conformational change of a GPCR through detection of a conformationally sensitive probe. In one embodiment, the conformationally sensitive probe is a detectable label, e.g. bound to a residue within the third loop (e.g., the third cytoplasmic loop) of the GPCR. In another embodiment, the conformationally sensitive probe is a protease cleavage site, where the accessibility of the site to cleavage changes depending upon the conformational of the GPCR (e.g., the conformation of the GPCR in the presence or absence of ligand).

Direct Labeling of GPCRs with a Detectable Probe.

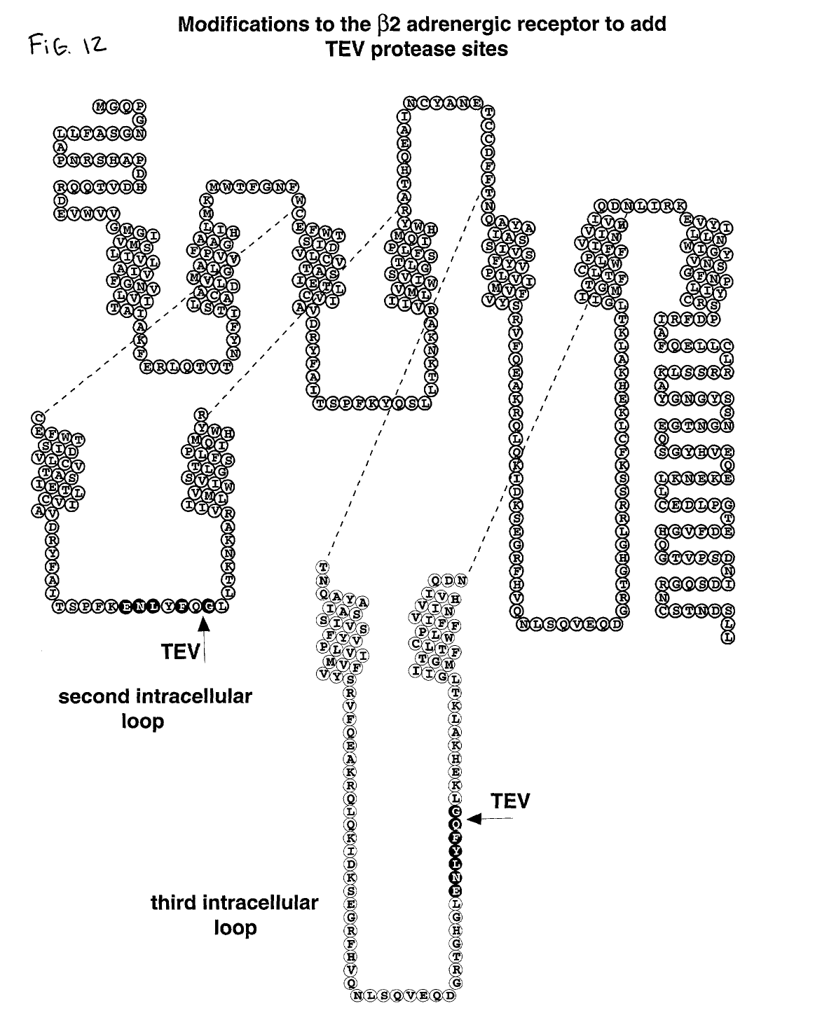

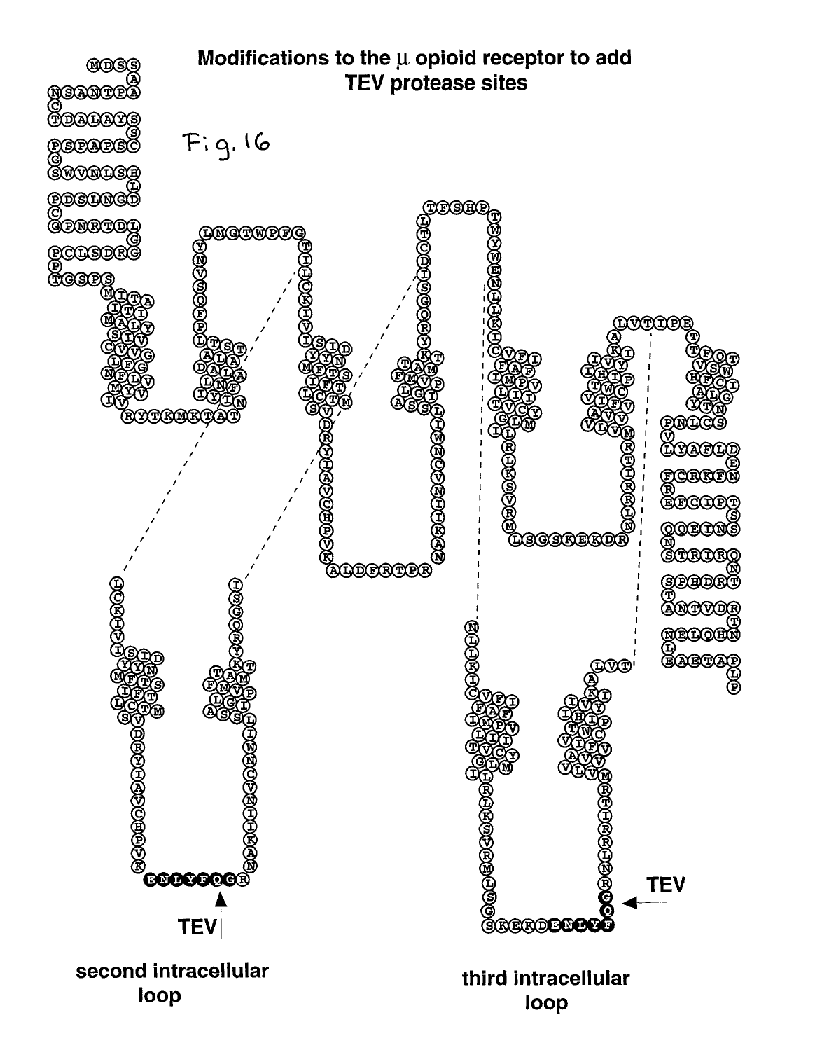

In one embodiment, the conformationally sensitive detectable probe is a detectable label that is attached to at least one amino acid residue of the GPCR in a conformationally sensitive structural domain of the GPCR, e.g., an amino acid residue of the third intracellular loop. In general, the amino acid residue(s) modified to contain or provide a conformationally sensitive detectable probe are those residues corresponding to: 1) the third intracellular loop conserved in GPCR proteins; 2) the second intracellular loop conserved in GPCR proteins; 3) amino acids in transmembrane helix 3 (TM3); and/or 4) amino acids in transmembrane helix 6 (TM6). These structural regions are conserved in GPCRs. Modified GPCRs include those modified to contain a conformationally sensitive detectable probe in one or more of these regions. Examples of modifications of two exemplary GPCRs, the β₂-AR and the μ opioid receptor, are illustrated in the Examples below and in FIGS. 12 and 16.

Various detectable labels include radioisotopes, fluorophores, chemiluminescers, nitroxide spin labels or other label that provides a change in detectable signal upon a change in conformation of the GPCR. Fluorescent labels are preferred detectable labels.

The purified, detectably labeled GPCR can be studied in detergent solution or fixed to a substrate such as a glass slide or an immobilized membrane (e.g., lipid bilayer, micelles, inside-out vesicles, and the like). Interaction of a ligand with the GPCR causes a conformational change in the receptor, which in turn changes the detectable signal (e.g., increase or decrease the signal) from the conformationally sensitive detectable probe. Ligand-induced changes in intensity of the detectable probe can be studied using conventional methods, e.g., fluorimeters or array readers. The change in detectable signal upon interaction of the detectably labeled GPCR with a ligand can be used to, for example, assess the affinity of the ligand for the receptor. In addition or alternatively, where the GPCRs are provided on an array (or the ligands are provided on an array), the change in detectable signal at a location(s) on the array, as well as the relative amount of change in the detectable signal, can be used to identify GPCR-ligand interactions, and provide for identification of the corresponding GPCR (or ligand) on the array by virtue of the assigned array coordinates.

Modifications to Modulate Assay Output

In some embodiments, the assay can be modified to enhance detection of ligand-GPCR binding. For example, in some embodiments, the detectable signal will not change upon ligand binding to the GPCR. However, the addition of reagents (e.g., fluorescence quenchers) that partition into specific environments around the receptor (e.g., within the aqueous environment or within the lipid bilayer) can be used to reveal conformational changes that occur upon receptor-ligand interactions. Exemplary fluorescent quenching agents include, but are not necessarily limited to, the nitroxide labeled fatty acid (CAT-16), 5-doxyl stearate (5-DOX), potassium iodide (KI), and the like. In this embodiment, induction of a conformational change in the GPCR upon ligand binding results in movement of the detectable label (e.g., fluorophore) toward or away from a quenching reagent, thus modifying the detectable signal.

For example, where the detectable label is a fluorescent label, the detectable signal can be enhanced by adding a quenching agent to the detergent micelle or to the lipid bilayer. For example, CAT-16 is a modified fatty acid has a nitroxide spin label covalently attached to the polar head group. Studies on β2-AR labeled with fluorescein at Cys265 show that agonist-induced changes in fluorescence are enhanced in the presence of CAT16, suggesting that agonist-induced structural changes lead to the movement of fluorescein on Cys265 closer to the polar surface of the detergent micelle. For some receptors, it may be necessary to modify one or more labeling site(s) for the fluorophore to obtain optimal signal. Thus, modified receptors having reactive cysteines at positions −2, −1, +1 and +2 relative to the position homologous to Cys265 in the β2-AR can be generated

To improve the signal to noise, a second detectable probe (e.g. a second fluorescent probe having a different excitation and emission spectrum) can be added to a conformationally insensitive domain on the receptor. The detectable signal of the second detectable probe would be used to control for variations in signal intensity due to differences in the amount of receptor protein. The signal would therefore be, for example, the ratio of conformationally sensitive probe (Ps) to the conformationally insensitive probe (Pi). The intensity of Ps will change when the receptor is bound to agonists and partial agonists, but will not change when the receptor is bound to antagonists. Antagonist binding can, however, be detected by stabilization of receptor against denaturation by reducing agents.

Modification of GPCRs useful in the Invention

GPCRs modified to have an amino acid residue within a conformationally sensitive domain and suitable for attachment to a detectable label are within the scope of the invention. For example, where the GPCR to be analyzed does not have an amino acid residue analogous to the cysteine residue at position 265 of β2-AR, the GPCR can be modified using available recombinant techniques to introduce such a cysteine residue (e.g., using site-specific mutagenesis or other available techniques). Alternatively, the GPCR to be analyzed can have an intracellular loop analogous to the third intracellular loop of β2-AR replaced with the third intracellular loop of the β2-AR.

GPCRs of interest can be modified using standard recombinant DNA technology to include an epitope tag at the amino terminal end, carboxyl terminal end, or both. For example, a GPCR can be modified to have an amino terminal FLAG epitope and a carboxyl terminal hexahistidine sequence. These modifications facilitate purification of the protein. In addition, the intracellular domains of the receptors can be modified so that all native cysteines, other than the consensus palmitoylation sites, are mutated to serine or alanine.

In one embodiment, a cysteine can be added to the cytoplasmic end of TM6 corresponding to Cys265 in the human β2-AR. This can also be accomplished by an exchange of the entire third intracellular loop of the GPCR for the third intracellular loop of the β2AR. The modified GPCRs can be expressed in insect cells or other host cells using standard recombinant methods.

After sufficient time for GPCR production, cells are harvested and intact cells are treated with iodoacetamide to block native cysteines in the extracellular domains of the GPCR. This will prevent nonspecific labeling of these sites with the fluorescent probe. Cells are then lysed, and membranes prepared. The membranes can be frozen for years (e.g. at −80° C.). Receptors can purified by chromatography on Flag affinity resin where the Flag epitope is used. The purified receptor is then labeled with fluorescein (or another environmentally sensitive fluorophore) and the unreacted fluorophore is separated from the labeled protein using Ni chelating chromatography.

Using Site-Specific Proteases to Monitor Ligand-Induced Changes in Receptor Structure.

Ligand-induced changes in the conformation of the β₂-AR alter its susceptibility to several proteases. This property, when coupled with a highly selective protease, can be used to detect ligand-induced conformational changes.

For each GPCR, a cleavage site for a highly specific recombinant protease, such as the tobacco etch virus (TEV) protease, is introduced into the third intracellular loop near the cytoplasmic end of TM6. An alternative site is within the second intracellular loop. Conformational changes induced by ligand binding result in movement of these intracellular loops, thereby altering accessibility of the protease to the cleavage site.

Introduction of Protease Cleavage Sites into GPCR

Conformational assays can be based on a change in the accessibility of an introduced protease cleavage site. In some embodiments it may be desirable to introduce multiple such cleavage sites.

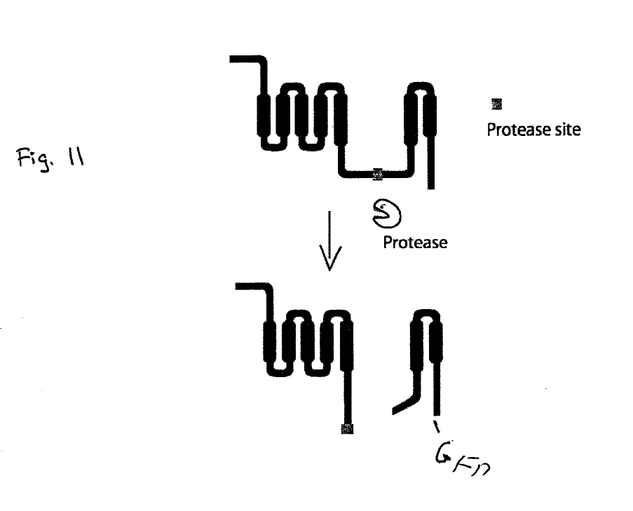

In general, the GPCR is modified to have a protease cleavage site introduced at a position so that ligand binding results in an alteration of the accessibility of the cleavage site to protease cleavage, e.g., within a loop that changes in conformation during ligand interaction. In general, the protease cleavage sits is positioned within the third intracellular loop of the GPCR. FIG. 11 provides a schematic of a GPCR having a protease cleavage site within the third intracellular loop.

Exemplary cleavage sites that can be introduced into the modified GPCRs of the invention include, but are not limited to, trypsin, chymotrypsin, pepsin, elastase, pronase, endoproteases (e.g., Arg-C, Asp-C, Glu-C, and Lys-C), endopeptidases such as Hepatitis C virus NS3 endopeptidase, tobacco etch virus, and factor Xa proteases. Methods for use of proteases in the cleavage of protease cleavage sites are well known in the art.

Detection of Conformational GPCR Changes using Protease as a Probe

Detection of protease cleavage products in conformational assays using protease cleavage of a protease cleavage site in the GPCR can be accomplished in a variety of ways. Exemplary methods for detection of cleavage products include, but are not necessarily limited to: 1) detection of the cleavage product that is produced from the N-terminal portion of the GPCR; 2) detection of the cleavage product that is produced from the C-terminal portion of the GPCR; 3) assaying for a new epitope created at an introduced cleavage site following protease action; 4) assaying for the disappearance of an epitope that is present at the cleavage site prior to cleavage; and 5) where the GPCR is modified to have two protease cleavage sites flanking an epitope tag, detection of the released epitope tag. Detection of changes at the protease cleavage site are preferred over detection of N-terminal or C-terminal cleavage products. Other variations will be readily apparent to the ordinarily skilled artisan.

Epitope Tags

In one embodiment, the GPCR is modified to include an epitope to facilitate detection (e.g., for detection of a protease cleavage product by detection of an epitope), anchoring of the GPCR to a substrate (e.g., by binding to an anti-epitope antibody), or both. In general, such modified proteins comprise a heterologous epitope domain. By “heterologous” is meant that the two elements are derived from two different sources, e.g., the resulting chimeric protein is not found in nature. A variety of epitopes may be used to tag a protein, so long as the epitope (1) is heterologous to the naturally-occurring GPCR, and (2) the epitope-tagged GPCR retains at least part and preferably all of the biological activity of the native GPCR, particularly with respect to the conformational change that occurs upon ligand interaction. Such epitopes may be naturally-occurring amino acid sequences found in nature, artificially constructed sequences, or modified natural sequences.

A variety of artificial epitope sequences are suitable for use as epitope tags in the present invention. In general, any epitope tag useful for tagging and detecting recombinant proteins may be used in the present invention. One such tag, the eight amino acid FLAG marker peptide (Asp-Tyr-Lys-Asp-Asp-Asp-Asp-Lys) (SEQ ID NO: 1), has a number of features which make it particularly useful for not only detection but also affinity purification of recombinant proteins (Brewer (1991) Bioprocess Technol. 2:239-266; Kunz (1992) J. Biol. Chem. 267:9101-9106). A further advantage of the FLAG system is that it allows cleavage of the FLAG peptide from purified protein since the tag contains the rare five amino acid recognition sequence for enterokinase. Additional artificial epitope tags include an improved FLAG tag having the sequence Asp-Tyr-Lys-Asp-Glu-Asp-Asp-Lys (SEQ ID NO: 2), a nine amino acid peptide sequence Ala-Trp-Arg-His-Pro-Gln-Phe-Gly-Gly (SEQ ID NO: 3) referred to as the “Strep tag” (Schmidt (1994) J. Chromatography 676:337-345), poly-histidine sequences, e.g., a poly-His of six residues which is sufficient for binding to IMAC beads, an eleven amino acid sequence from human c-myc recognized by monoclonal antibody 9E10, or an epitope represented by the sequence Tyr-Pro-Tyr-Asp-Val-Pro-Asp-Tyr-Ala-Ile-Glu-Gly-Arg (SEQ ID NO: 4) derived from an influenza virus hemagglutinin (HA) subtype, recognized by the monoclonal antibody 12CA5. Also, the Glu-Glu-Phe sequence recognized by the anti- tubulin monoclon al antibody YL1/2 has been used as an affinity tag for purification of recombinant proteins (Stammers et al. (1991) FEBS Lett. 283:298-302).

Exemplary Assays for Detection of Protease Cleavage Products

As described generally above, detection of conformational changes in GPCRs by detection of accessibility of a protease cleavage site can be accomplished in a variety of ways. Wherein the GPCR has a single protease cleavage site, the GPCR is contacted with a candidate agent (e.g., either in a cell-free or cell-based assay), and with protease that can cleave the protease cleavage site of the GPCR. If the candidate agent is, for example, an agonist of the GPCR, the agent binds to the GPCR and induces a conformational change that alters the accessibility of the protease cleavage site to cleavage by the protease.

At this point the assay has up to three different polypeptides present: 1) intact, uncleaved GPCR (e.g., GCPR that is not bound by agonist); 2) a protease cleavage product produced from the N-terminal portion of the GPCR; and 3) a protease cleavage product produced from the C-terminal portion of the GPCR. In one embodiment, the GPCR is immobilized on a substrate by attachment at the C-terminus (e.g., by binding to an anti-C-terminal GPCR antibody that is in turn bound to a substrate). Detection of protease cleavage can then be accomplished by detection of a N-terminal GPCR cleavage product released from the bound GPCR. Detection of an increased level of N-terminal GPCR cleavage product in the supernatant indicates the candidate agent is a GPCR ligand that induces a conformational change in the GPCR. Conversely, candidate agent activity in GPCR binding can be detected by a decrease in detection of N-terminal GPCR bound to the substrate.

Alternatively, the GPCR can be bound to a substrate by the N-terminal end, and a conformational change in the GPCR due to interaction with the candidate agent can be detected by detection of a released N-terminal GPCR cleavage product. Conversely, candidate agent activity in GPCR binding can be detected by a decrease in C-terminal GPCR bound to the substrate.

In one embodiment, the disappearance of a epitope that is normally present in the GPCR prior to cleavage can serve as the basis for the assay. For example, the uncleaved GPCR may have to be modified to have an epitope that can be detected by an antibody, which epitope flanks or encompasses the protease cleavage site. Action of the protease on the cleavage site disrupts the epitope so that it is not detectable in the cleaved GPCR.

In another embodiment, the action of the protease at the introduced cleavage site is detected by detecting an epitope newly created by the action of the protease.

For example, the new epitope can be the newly created C-terminus generated by the protease at the cleavage site.

In another embodiment, the GPCR is modified to have two protease cleavage sites flanking an epitope tag. Binding of the GPCR to an agent having, for example, GPCR agonist activity, causes a conformational change that renders the protease cleavage sites accessible to the protease. Protease cleavage in turn results in liberation of the epitope tag. Detection of the released epitope tag indicates that the GPCR has undergone a conformational change, and that the candidate agent has activity in binding GPCR.

All assays can be conducted with an appropriate control, which can be performed in parallel. For example, the level of cleavage product production can be compared to that produced by contacting the GPCR with a known agonist of the GPCR.

Identification and Design of Therapeutic Compounds

A major asset of the invention is its ability to vastly increase, over current methods, the rate at which compounds can be evaluated for their ability to act as agonists, antagonists, and/or inverse agonists for GPCRs. As additional GPCR genes are identified and characterized, the activity of these receptors in response to various compounds, as well as to methods such as site directed mutagenesis, can be used to gain detailed knowledge about the basic mechanisms at work in these receptors. A fundamental knowledge of the basic mechanisms at work in these receptors will be of great use in understanding how to develop promising new drugs and/or to identify the fundamental mechanisms behind specific tastes, smells and the like.

GPCR-binding compounds identified by their induction of a conformational change according to the invention can be further screened for agonistic or antagonist action in other assays, e.g., in a functional assay that monitors a biological activity associated with GPCR function such as effects upon intracellular levels of cations (e.g., calcium) in a host cell, calcium-induced reporter gene expression (see, e.g., Ginty 1997 Neuron 18:183-186), or other readily assayable biological activity associated with GPCR activity. Such a functional assay can be based upon detection of a biological activity of the GPCR that can be assayed using high-throughput screening of multiple samples simultaneously, e.g., a functional assay based upon detection of a change in fluorescence which in turn is associated with a change in GPCR activity. Such functional assays can be used to screen candidate agents for activity as GPCR receptor agonists or antagonists.

Identification of Ligands for Orphan GPCRs

An assay system according to the invention can also be used to classify compounds for their effects on G protein coupled receptors, such as on orphan receptors, to identify candidate ligands that are the native ligands for these orphan receptors. Membranes having a modified orphan GPCR can be exposed to a series of candidate ligands, and the ligands with the ability to induce a conformational change upon the GPCR.

Identification of GPCRs Involved in Various Biological Processes

The GPCRs that are involved in biological responses, both normal responses (e.g., taste, smell, etc.) and pathological responses (e.g., the biological response to a GPCR involved in a disease or disorder) can be determined using assays of the invention. An assay using an array of membranes or micelles, each sample of the array having a modified GPCR, can be exposed to the stimulus (e.g., the odor, flavor compound, disease related complex, and the like), and any conformational change in the GPCR detected. This can identify multiple receptors in a high-throughput manner that are involved in the transduction of signals in response to the stimulus.

For example, the high-throughput assays of the invention can be especially useful in determining the spectrum of GPCRs, e.g., olfactory receptors, that are activated or inverse agonized by a specific substance or mixture of substances. For example, a liquid can be contacted with an array of membrane preparations having modified GPCR, and the GPCRs that undergo a conformational change identified.

Automated Screening Methods

The methods of the present invention may be automated to provide convenient, real time, high volume methods of screening compounds for GPCR ligand activity, or screening for the presence of GPCR ligand in a test sample. Automated methods are designed to detect changes in GPCR activity (e.g., via measurement of AC) over time (i.e., comparing the same apparatus before and after exposure to a test sample), or by comparison to a control apparatus which is not exposed to the test sample, or by comparison to pre-established indicia. Both qualitative assessments (positive/negative) and quantitative assessments (comparative degree of translocation) may be provided by the present automated methods.

An embodiment of the present invention includes an apparatus for determining GPCR response to a test sample. This apparatus comprises means, such as a fluorescence measurement tool, for measuring change in activity of a GPCR in response to a particular ligand. Measurement points may be over time, or among test and control GPCRs. A computer program product controls operation of the measuring means and performs numerical operations relating to the above-described steps. The preferred computer program product comprises a computer readable storage medium having computer-readable program code means embodied in the medium. Hardware suitable for use in such automated apparatus will be apparent to those of skill in the art, and may include computer controllers, automated sample handlers, fluorescence measurement tools, printers and optical displays. The measurement tool may contain one or more photodetectors for measuring the fluorescence signals from samples where fluorescently detectable molecules are utilized. The measurement tool may also contain a computer-controlled stepper motor so that each control and/or test sample can be arranged as an array of samples and automatically and repeatedly positioned opposite a photodetector during the step of measuring fluorescence intensity.

The measurement tool is preferably operatively coupled to a general purpose or application specific computer controller. The controller preferably comprises a computer program produce for controlling operation of the measurement tool and performing numerical operations relating to the above-described steps. The controller may accept set-up and other related data via a file, disk input or data bus. A display and printer may also be provided to visually display the operations performed by the controller. It will be understood by those having skill in the art that the functions performed by the controller may be realized in whole or in part as software modules running on a general purpose computer system. Alternatively, a dedicated stand-alone system with application specific integrated circuits for performing the above described functions and operations may be provided.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

Methods and Materials

The following methods and materials were used in Examples 1-5 below.

Construction, expression and purification of the β2 adrenergic receptor. Construction, expression and purification of human β2AR were performed as described (Ghanouni, P et al., J Biol Chem 275:3121-3127 (2000)). Mutations Glu224Lys, Cys378Ala, and Cys406Ala (where the first amino acid indicates the native residue, the number indicates the residue position, and the second amino acid represents the amino acid substituted for the native amino acid) were all generated on a background in which all of the lysines in the receptor had been mutated to arginine (Parola, A. L. et al., Anal Biochem 254:88-95(1997)). A sequence coding for the cleavage site for the Tobacco Etch Virus (TEV) protease (Gibco-BRL) was added to the 5′ end of the receptor construct via the linker-adapter method. All mutations were confirmed by restriction enzyme analysis and sequenced. The mutant receptor demonstrated only minor alterations in the general pharmacological properties of the receptor, as assessed by the affinity of the mutant receptor for isoproterenol and alprenolol (KI for ISO=150±40 μM for mutant receptor vs. 210±21 μM for wildtype (Seifert, R., et al., J Biol Chem 273:5109-16(1998)); KD for ALP=4.3±0.6 nM for mutant receptor vs. 1.7±0.9 nM for wildtype (Gether, U. et al., J Biol Chem 270, 28268-75 (1995)).

Fluorescent Labeling of Purified β2 Adrenergic Receptor. Purified, detergent soluble receptor was diluted to 1 μM in HS buffer (20 mM Tris, pH 7.5, 500 mM NaCl, 0.1% n-dodecyl maltoside (NDM)) and reacted with 1 μM fluorescein maleimide (FM; Molecular Probes) for 2 h on ice in the dark. The reaction was quenched with the addition of 1 mM cysteine. The receptor was bound to a 250 μl Ni-chelating sepharose column and the column was washed alternately with 250 μl HS buffer and 250 μl NS buffer (20 mM Tris, pH 7.5, 0.1% NDM) for a total of ten cycles to remove free FM. The labeled protein (FM-β2AR) was eluted with HS buffer with 200 mM imidazole, pH 8.0. FM-β2AR was diluted approximately 1:100 in HS buffer for fluorescence measurements. Fluorescence in control samples without receptor was negligible. The labeling procedure resulted in incorporation of 0.6 mol of FM per mol of receptor, based on an extinction coefficient of 83,000 M-1 cm-1 for FM and a molecular mass of 50 kDa for the β2AR.

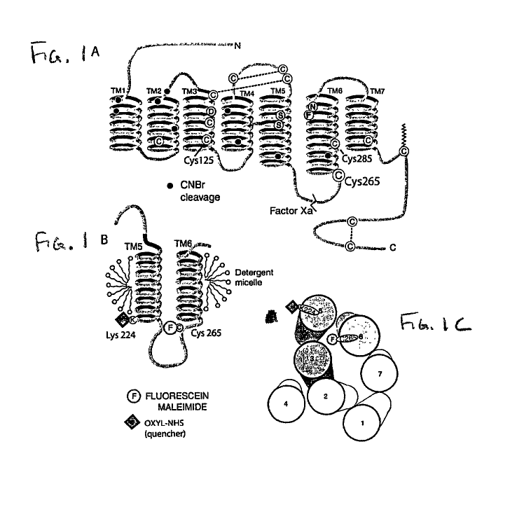

For labeling the Q224K site on the mutant receptor, the sample was split after labeling with FM (1 h) and dialyzed for 1 h at room temperature into a Hepes HS buffer. Half of the sample was treated with 1 mM oxyl-NHS for 1 h on ice. Both the FM alone and the FM+oxyl-NHS samples were then treated with TEV protease (Gibco-BRL) according to the manufacturer's instructions and then washed on a Ni-chelating sepharose column as above. Equivalent amounts of FM- and FM+oxyl-NHS-labeled receptor, as confirmed by protein assay (Bio-Rad DC Kit), were thus prepared for comparison. The TEV protease site at the N-terminus of the receptor allowed us to remove any probe located at the N-terminus after labeling the receptor with an amine-reactive tag. The location of the FM labeling site at Cys265 in both the wildtype and mutant receptors was verified by peptide mapping with protease factor Xa and cyanogen bromide. Cleavage sites are as indicated in FIG. 1.

Fluorescence spectroscopy. Experiments were performed on a SPEX Fluoromax spectrofluorometer with photon counting mode using an excitation and emission bandpass of 4.2 nm. Approximately 25 pmol of FM-labeled β2 adrenergic receptor were used in 500 μl of HS buffer. Excitation was at 490 nm and emission was measured from 500 to 599 nm with an integration time of 0.3 s/nm for emission scan experiments. For time course experiments, excitation was at 490 nm and emission was monitored at 517 nm. For studies measuring ligand effects, no difference was observed when using polarizers in magic angle conditions. Unless otherwise indicated, all experiments were performed at 25° C. and the sample underwent constant stirring. Fluorescence intensity was corrected for dilution by ligands in all experiments and normalized to the initial value. All of the compounds tested had an absorbance of less than 0.01 at 490 and 517 nm in the concentrations used, excluding any inner filter effect in the fluorescence experiments.

Fluorescence lifetime determination. Fluorescence lifetime measurements of the FM-labeled β2 adrenergic receptor were carried out using a PTI Laserstrobe fluorescence lifetime instrument. Measurements were taken at 25° C., using 490 nm excitation pulses (fill width half maximum (FWHM)˜1.4 ns) to excite the samples, and emission was monitored through a combination of three >550 nm long pass filters. Measurements used 225 μl of a 5 μM sample placed in a 4×4 mm cuvette, and represent 3 average shots of 5 shots per point, collected in 150 channels. The fluorescence decays were fit to a single exponential using the commercial PTI program.

Quenching of fluorescence. To quench the fluorescence, FM was diluted to 1 μM in HS buffer. The dye was diluted into 375 μl of a buffer containing 20 mM HEPES, pH 7.5, and 0.1% NDM. Experiments were performed at the indicated concentration of potassium iodide, freshly made in 10 mM Na₂S₂O₃, while the total salt concentration was maintained at 250 mM with potassium chloride in all experiments. Potassium iodide and potassium chloride at concentrations up to 250 mM do not alter the ligand binding properties of the β2AR (Gether et al. (1995) J. Biol. Chem. 270:28268-75). For nitroxide quenching, receptor was diluted into HS buffer. Experiments were performed at the indicated concentration of nitroxide fatty acids (Molecular Probes), while maintaining total fatty acid concentration at 100 μM with stearic acid. After each addition of quencher, samples were thoroughly mixed, incubated for 10 min (KI) or 5 min (nitroxides), and fluorescence was recorded by exciting at 490 nm and performing an emission scan from 500-599 nm.

Data were plotted according to the Stem-Volmer equation, Fo/F=1+Ksv(KI), where Fo/F is the ratio of fluorescence intensity in the absence and presence of KI, and Ksv is the Stem-Volmer quenching constant. The Ksv values thus obtained were then used with the measured fluorescence lifetimes (τₒ) to determine the bimolecular quenching constant, kq (Ksv=kq·τₒ) (Lakowicz, J. R. (1983) Plenum Press, N.Y.). For quenchers, a time scan was initiated after the emission scan and 100 μM (−)-isoproterenol was added after 2 min. At 10 min, 20 μM (−)-alprenolol was added and the extent of reversal determined. The quenchers used did not alter the ability of (−)-isoproterenol or (−)-alprenolol to compete with (³H)DHA.

### Example 1

Effect of Full and Partial Agonists on Fluorescence of FM-β2AR Correlates with the Biological Properties of the Agonists.

The effect of full and partial agonists on the fluorescence of FM-β2AR correlated with the biological properties of the agonists. Only Cys265 was labeled when purified, detergent solubilized β2AR (1 μM) is reacted with fluorescein maleimide at a 1:1 stoichiometry. This polar fluorophore does not label transmembrane cysteines and the two other potentially accessible cysteines in the carboxyl terminus (FIG. 1A) form a disulfide bond during purification. The specificity of labeling was confirmed by peptide mapping studies with factor Xa (which cleaves only in the third intracellular loop) and cyanogen bromide (which cleavage at methionines, shown in FIG. 1A). When FMβ2AR is cleaved with factor Xa fluorescence labeling is only observed on the carboxyl terminal half of the protein. Following cleavage of FMβ2AR with cyanogen bromide labeling is localized to a 7 kDa peptide representing a portion of the third intracellular loop containing Cys 265 (data not shown). Labeling of the β2AR with fluorescein did not alter ligand binding or G protein coupling in a reconstitution assay (data not shown).

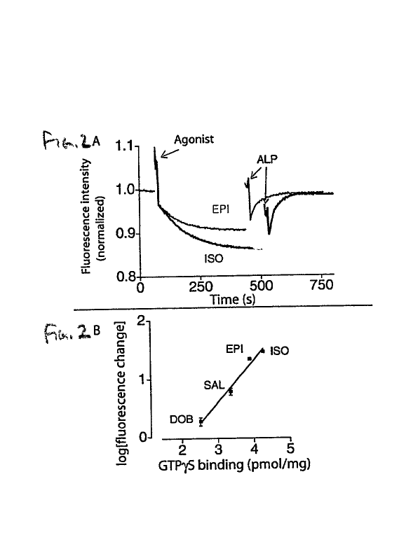

The fluorescence properties of FM-β2AR were examined by monitoring fluorescence as a function of time. As illustrated in FIG. 2A, the change in intensity of FM-β₂AR in response to the addition of the full agonist (−)-isoproterenol (ISO) and the strong partial agonist epinephrine (EPI) was reversed by the neutral antagonist (−)-alprenolol (ALP). All data represent experiments performed in triplicate. In most experiments, the ALP reversal was used to quantitate the magnitude of the agonist-induced change. The ALP reversal was found to be the most consistent measure for comparison of agonist-induced conformational changes because ALP reversal occurs over a shorter period of time relative to agonist responses and therefore is less subject to non-specific effects on fluorescence intensity (e.g., photobleaching, receptor denaturation) that affect the baseline. ALP alone did not induce any changes in fluorescence and treatment with ligands did not cause a change in the wavelength of maximum emission (data not shown). The partial agonists epinephrine (EPI), salbutamol (SAL) and dobutamine (DOB) produce progressively smaller changes in receptor fluorescence.

The agonist and partial agonist effects on the intensity of FM-β₂AR were compared with an assay of biological efficacy (GTPγS binding). FM-β₂AR was treated with different agonists and the change in fluorescence was measured at a time equal to 5 times the calculated t1/2 for each drug. All agonists were used at 100 mM in order to ensure saturation of the receptors and eliminate the effect of variations in agonist affinities. The ability of these ligands to stimulate GTPγS binding in a β₂AR-Gαs fusion protein was determined as previously described (Lee et al. (1999) Bichemistry 38:13801-9). All data represent experiments performed in triplicate. The magnitude of the effect of agonists on the fluorescence intensity of FM-β2AR correlates with the biological efficacy of these drugs in β2AR-mediated activation of Gs in membranes (FIG. 2B).

These experiments verify that fluorescence intensity changes in FM-β2AR reflect biologically relevant, ligand-induced conformational changes.

### Example 2

Kinetics of Agonist-Induced Conformational Change.

Rhodopsin has long been used as a model system for direct biophysical analyses of GPCR activation because of its natural abundance, inherent stability, and spectroscopically defined activation scheme (Sakrnar, T. P., Prog Nucleic Acid Res Mol Biol 59:1-34 (1998)). The recent crystal structure of bovine rhodopsin (Palczewski, K. et al., Science 289, 739-45 (2000)) provides the first high-resolution picture of the inactive state of this highly specialized GPCR. While the general features of this structure presumably apply across the broad family of GPCRs, the mechanism of rhodopsin activation is unique among GPCRs because of the presence of a covalent linkage between the receptor and its ligand, retinal. Thus, the dynamic processes of agonist association and dissociation common to the GPCRs for hormones, neurotransmitters, and other sensory stimuli are not part of the activation mechanism of rhodopsin. In contrast to rhodopsin, the β2 adrenergic receptor is activated by a functionally broad spectrum of diffusible ligands.

This difference between rhodopsin and the β2ARwais reflected in the rate of agonist-induced structural changes. Conformational changes induced in detergent-solubilized preparations of rhodopsin by light activation were very rapid, occurring with a t1/2 of milliseconds (Arnis et al., J Biol Chem 269, 23879-81(1994); Farahbakhsh, et al., Science 262, 1416-9 (1993)). In contrast, as shown in FIGS. 2A-2B, agonist activation of the β₂AR was slow, despite the rapid on-rate of agonist binding (t1/2˜20 sec) as calculated from the agonist affinity, the off-rate estimated from the alprenolol (ALP) reversal of the agonist effect (FIG. 2A) and the concentration of agonists used in these experiments (100 μM)). Under these conditions, the on-rate of agonist was comparable to the more rapid rate of reversal of the agonist effect by the antagonist alprenolol (t1/2 at 25° C.=22.8±3.6 s, Mean±S.E.M., n=3).

The same slow rate of agonist-induced conformational change was also observed with a different fluorescent reporter on Cys125 in TM3 and on Cys285 in TM6 of the β2AR (FIG. 1A) (Gether, U., Lin, S., Ghanouni, P., Ballesteros, J. A., Weinstein, H. & Kobilka, B. K. (1997) Embo J 16, 6737-47), and Salamon and colleagues observed a similar rate of agonist induced conformational changes in the α-opioid receptor analyzed by surface plasmon resonance spectroscopy (Salamon, Z. et al., Biophys J 79:2463-74 (2000)). Thus, agonist binding precedes the conformational change. The rate of conformational change is temperature dependent, with the rate at 37° C. approximately 3 times that at 25° C. (data not shown). The slow, temperature dependent rate of confromation change and the rpaid reversal suggests that the active state is a relatively high energy state which may be reached through one or jmore intermediate states, as illustrated in Equation 1:

$$A + R {{↔}^{{k}_{1}}}_{{k}_{2}} {AR}^{′} {{↔}^{{k}_{3}}}_{{k}_{4}} {AR}^{*} \qquad ( 1 )$$

where R is the inactive receptor, R′ is the agonist bound, inactive receptor and R* is the active receptor. k3 is predicted to be slow relative to k1, k2 and k4. Moreover the agonist binding site in R′ may not be identical to the binding site in R*. The ligand binding site for the β2AR has been well characterized by mutagenesis studies and lies relatively deep in the transmembrane domains (FIG. 1A). Without being held to theory, the difference in the rate of conformation change between rhodopsin and the β2AR can be attributed to the need for the ligand to diffuse into the binding pocket and the smaller energy associated with agonist binding.

### Example 3

Agonist-Induced Movement of FM Bound to Cys265 Relative to Molecular Landmarks.

To characterize the agonist-induced structural changes in the G protein coupling domain containing Cys265, agonist-induced changes in the interaction of FM-β2AR with a variety of fluorescence quenchers was examined.

The results of these experiments were interpreted in the context of a three dimensional model of the β2AR based on the recent crystal structure of rhodopsin in the inactive state. Based on a simplified model viewed from the cytoplasmic surface of the receptor, we would predict that in the absence of agonist, fluorescein bound to Cys265 would be facing the interior of a bundle of helices formed by the cytoplasmic extensions of TM3, TM5 and TM6 (FIG. 1C).

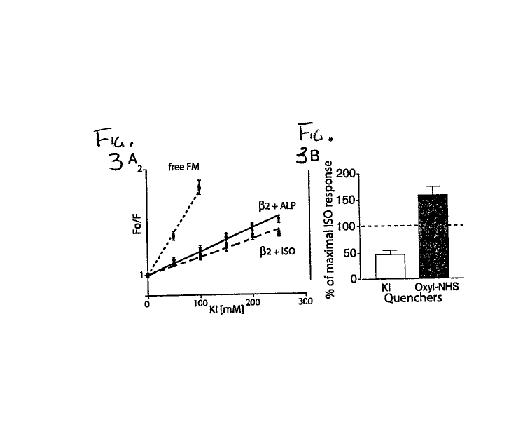

The accessibility of the water-soluble quencher potassium iodide to the fluorescein bound to Cys265 was then determined (FIG. 3A). KI was added to fluorescein maleimide reacted with cysteine, to labeled receptor incubated with 20 mM (−)-alprenolol, and to labeled receptor incubated with 100 mM (−)-isoproterenol. Fluorescence was measured and plotted as described in Methods. The quenching constant Kₛᵥ was 7.9±0.4 M⁻¹ for fluorescein alone, 2.19±0.06 M⁻¹ for labeled receptor incubated with (−)-alprenolol, and 1.66±0.06 M⁻¹ for labeled receptor incubated with (−)-isoproterenol. The difference between isoproterenol and alprenolol was significant (p<0.05, unpaired t test). There was no difference in Kbetween buffer alone and alprenolol treatments. All values are Mean±S.E.M., n=3. The results are shown in FIG. 3A.

The effect of quenchers KI and Oxyl-NHS on the magnitude of the ISO-induced decrease in fluorescence was also determined (FIG. 3B). “% of control ISO response” was calculated using the formula [100(ISO induced change in fluorescence in the presence of quencher)/(ISO induced change in fluorescence in the absence of quencher)]. For the aqueous quencher KI, the ISO-induced change in fluorescence in the presence of 250 mM KI was less than that in the presence of 250 mM KCl (55.4±8.3% of control ISO response). (In contrast to the aqueous quencher KI, covalent binding of the spin-labeled quencher Oxyl-NHS to K224 in TM5 increased the magnitude of the ISO response relative to the control (158±8% control ISO response), see below). In these experiments, the magnitude of the ALP reversal of the ISO-induced change in fluorescence was used as a measure of the magnitude of the ISO response. The results are shown in FIG. 3B. All values are Mean±S.E.M., n=3.

As represented in the Stem-Volmer plot (FIG. 3A), steady-state fluorescence quenching by KI is much lower for fluorescein bound to the receptor when compared to fluorescein maleimide bound to free cysteine in solution. This indicates that the fluorescein site on the receptor is relatively inaccessible to the water soluble quencher KI, as expected based on the predicted position of the fluorescein bound to Cys265 (FIG. 1C).

To determine the effect of agonist on KI quenching, we measured the fluorescence lifetimes of FM-β2AR in the presence ISO and ALP, which permitted us to calculate the bimolecular quenching constant (kq=Ksv/τₒ) using the average value of the lifetime of FM-β2AR in the presence of either ISO (kq=0.45±0.01×10-9 M-1s-1) or ALP (kq=0.51±0.01×10-9 M-1s-1). There was no difference between the extent of KI quenching in the ligand-free or ALP-bound receptor. However, the lower kq in the ISO bound state clearly shows that the fluorescein label on the β2AR was less accessible to the water-soluble quenching reagent KI in the presence of the agonist ISO (Dunham and Farrens J Biol Chem 274:1683-90 (1999)). As a result, the magnitude of the ISO-induced change in fluorescence in the presence of 250 mM KI was smaller than in the presence of 250 mM KCI (FIG. 3B). Thus, ISO induces a conformational change which enhances the intra-receptor quenching of FM bound to Cys265, but reduces access of Cys265 to exogenous, aqueous quencher KI. The burial of Cys265 away from the aqueous milieu could be accomplished by a movement of TM6 toward the membrane (FIG. 1B) and/or by a movement of TM6 that would bring Cys265 closer to either TM3 or TM5 (FIG. 1C).

### Example 4

Agonist-Induced Movement of Cys265 Relative to Lys224.

To distinguish between the movement of Cys265 toward either TM3 or TM5, a modified β2AR that permits site-specific attachment of an amine-reactive, spin-labeled quencher at the cytoplasmic border of TM5 was generated (FIG. 1C). In order to position the quencher at the base of TM5, the template β2AR was used in which all of the lysines have been replaced by arginine (Parola et al., Anal Biochem 254, 88-95 (1997)) and changed Glu224 to lysine. This mutant was purified and studied the interaction between FM at Cys265 and oxyl-NHS at Lys224.

While the baseline quenching of FM on Cys265 with oxyl-NHS bound to Lys224 was less that 10%, the effect of ISO on decreasing of FM fluorescence intensity (as reflected in the magnitude of the ALP reversal) was enhanced by more than 50% with the quencher bound to Lys224 (FIG. 3B). Since the effect of this quencher was distance dependent, the increase in the extent of quenching reflects an agonist-induced conformational change which brings these regions of TM6 and TM5 closer together.

### Example 5

Agonist Induces Movement of FM Bound to Cys265 Relative to a Lipophilic Quencher in the Detergent Micelle.

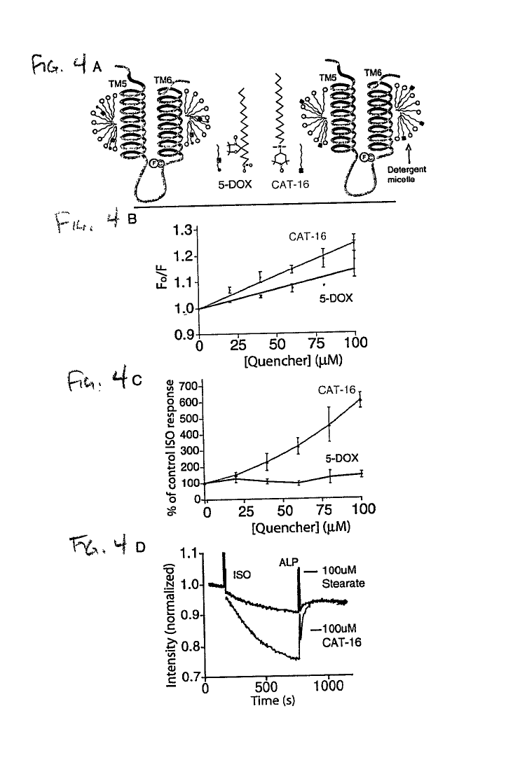

Due to the location of the fluorophore close to the predicted protein-lipid interface (FIG. 1B) of TM6, the interaction between the fluorophore and nitroxide spin-labeled fatty acids which partition into the detergent micelle was used to observe relative motion between the Cys265 and the micelle (FIG. 4A). FIG. 4A is a schematic depicting the structure of CAT-16 and 5-doxyl stearate (5-DOX), as well as the putative location of these quenching groups in the micelle. The quenching group on CAT-16 is localized on the polar surface of the micelle. The quenching group on 5-DOX is located within the hydrophobic core of the micelle.

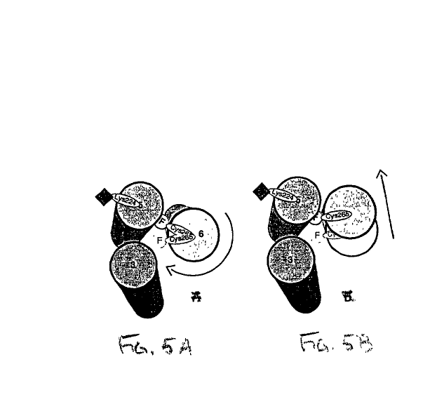

FIG. 4B provides a Stern-Volmer plot depicting the extent of quenching of FM-b2 AR by increasing concentrations of CAT-16 or 5-DOX. Quenchers were added to labeled receptor and fluorescence was measured and plotted as in FIG. 3 and Methods. The total lipid concentration was kept constant at 100 mM with stearic acid. The quenching constant Ksv was 2.4±0.1 mM⁻¹ in the presence of CAT-16 and 1.4±0.2 mM⁻¹ in the presence of 5-DOX. FIG. 5C shows the differing effects of CAT-16 and 5-DOX on agonist-induced fluorescence change of FM-b2 AR. The extent of response to (−)-isoproterenol is presented as a % control ISO response, calculated as in FIG. 3. FIG. 5D is an example of the experiments used to generate the ratios in FIG. 4c. In this example, FM-β2 AR was incubated with either 100 mM CAT-16 or with 100 mM stearic acid. The response to agonist was monitored as described for the experiment depicted in FIG. 2. In the presence of the quencher CAT-16, (−)-isoproterenol induced a 24.2±0.3% decrease in fluoresence versus 4.1±10.6% in the presence of the stearic acid. All values are Mean±S.E.M., n=3.

Because of their ability to quench the excited state of a variety of fluorophores in a distance-dependent manner, these spin-labeled fatty acid derivatives have been used extensively to study the distribution, location and dynamics of fluorescently tagged proteins and lipids (Matko, J. et al, Biochemistry 31, 703-11 (1992)). Fatty acid derivatives with spin labels at two different locations along the carbon chain were examined (FIG. 4A) and observed the best quenching of fluorescein by CAT-16, which has a charged spin label on the head group of the fatty acid (FIG. 4B). The magnitude of the change in fluorescence intensity of FM-β2AR in response to the agonist ISO is dramatically increased in the presence of CAT-16 compared to the control fatty acid stearate (FIG. 4c). This effect was not observed with 5-DOX (FIG. 4C). For example, 100 μM 5-DOX quenched baseline fluorescence by 12% (FIG. 4B), but had no significant effect on the magnitude of the agonist-induced change in fluorescence (FIG. 4C). In contrast, 50 μM CAT-16 produced a similar (˜12%) quenching in baseline fluorescence (FIG. 4b), but increased the magnitude of the agonist-induced fluorescence change by more than two fold (FIG. 4c). This indicates that ISO induces a conformational change at Cys265 which brings the fluorophore closer to the nitroxide spin label of CAT-16 in the detergent micelle border, but not significantly closer to nitroxide spin label in 5-DOX, which would be buried within the hydrophobic core of the micelle. According to the models shown in FIG. 4a and FIG. 5, a piston-like movement of TM6 into the detergent micelle would bring fluorescein closer to the quenchers on both 5-DOX and CAT-16, but a clockwise rotation of TM6 and/or a tilting of TM6 would bring fluorescein closer to CAT-16 without significantly changing its position relative to 5-DOX.

### Examples 6-9

Functionally Different Agonists Induce Distinct Conformations in the G Protein Coupling Domain of β2AR Methods and Materials

The following methods and materials were used in Examples 6-9.

Fluorescence spectroscopic studies of the β₂AR. Construction, expression and purification of human β₂AR were performed as described (Gether, et al. (1995) J Biol Chem 270(47), 28268-75). For labeling, purified, detergent-solublized wild-type receptor was diluted to 1 μM in HS buffer (20 mM Tris, pH 7.5, 500 mM NaCl, 0.1% n-dodecyl maltoside (NDM)) and reacted with 1 μM fluorescein maleimide (FM; Molecular Probes) for 2 h on ice in the dark. The reaction was quenched with the addition of 1 mM cysteine. The receptor was bound to a 250 μl Ni-chelating sepharose column and the column was washed alternately with 250 μl HS buffer and 250 μl NS buffer (20 mM Tris, pH 7.5, 0.1% NDM) for a total of ten cycles to remove free FM. The labeled protein (FM-β₂AR) was eluted with HS buffer with 200 mM imidazole, pH 8.0. FM-β₂AR was diluted approximately 1:100 in HS buffer for fluorescence measurements. Fluorescence in control samples without receptor was negligible.

The stoichiometry of labeling was determined by measuring absorption at 490 nm and using an extinction coefficient of 83,000 M⁻¹ cm⁻¹ for FM and a molecular mass of 50 kDa for the β₂AR. The labeling procedure resulted in incorporation of 0.6 mol of FM per mol of receptor. Fluorescence spectroscopy experiments were performed on a SPEX Fluoromax spectrofluorometer with photon counting mode using an excitation and emission bandpass of 4.2 nm. Approximately 25 pmol of FM-labeled β₂ adrenergic receptor was diluted into 500 μl of 200 mM Tris, pH 7.5, 500 mM NaCl, 0.1% NDM, 100 mM mercaptoethanolamine (MEA). Excitation was at 490 nm and emission was measured from 500 to 599 nm with an integration time of 0.3 s/nm for emission scan experiments.

For time course experiments, excitation was at 490 nm and emission was monitored at 517 nm. For anisotropy studies, fluorescence intensities were measured with excitation and emission polarizers in horizontal (H) and vertical (V) combinations. The G factor was calculated from the ratio of the intensities (I) of I_{HV}/I_{HH} and the anisotropy (r) was calculated from

$$r = ( \frac{{I}_{VV} - {GI}_{VH}}{{I}_{VV} + 2 {GI}_{VH}} ) .$$

For studies measuring ligand effects, no difference was observed when using polarizers in magic angle conditions. Unless otherwise indicated, all experiments were performed at 25° C. and the sample always underwent constant stirring. The volume of the added ligands was 1% of total volume, and fluorescence intensity was corrected for this dilution in all experiments shown. All of the compounds tested had an absorbance of less than 0.01 at 490 and 517 nm in the concentrations used, excluding any inner filter effect in the fluorescence experiments.

Fluorescence lifetime analysis of fluorescein labeled β₂AR. To determine fluorescence lifetimes, approximately 250 pmol FM-β₂AR was diluted in 1.5 ml of 200 mM Tris, pH 7.5, 500 mM NaCl, 0.1% NDM, 100 mM MEA and incubated for 10 min at 25° C. with or without ligand. Fluorescence lifetimes were measured using a frequency-domain 10 GHz fluorometer equipped with Hamamatsu 6 μm microchannel plate detector (MCP-PMT) as previously described (Laczko, et al. (1990) Rev. Sci. Instrum. 61, 2331-2337). The instrument covered a wide frequency range (4-5000 MHz), which allowed detection of lifetimes ranging from several nanoseconds to a few picoseconds. Samples were placed in a 10-mm path-length cuvette. The excitation was provided by the frequency-doubled output of a cavity-dumped pyridine-2 dye laser tuned at 370 nm synchronously pumped by a mode-locked argon ion laser. Sample emission was filtered through Coming 3-72 and 4-96 filters. For the reference signal, DCS in methanol (463 ps fluorescence lifetime) was observed through the same filter combination.

The governing equations for the time-resolved intensity decay data were assumed to be a sum of discrete exponentials as in

$$I ( t ) = {I}_{o} {\sum }_{i} {α}_{i} {ⅇ}^{t / {τ}_{i}} ,$$

where I(t) is the intensity decay, αᵢ is the amplitude (pre-exponential factor) and τᵢ is the fluorescence lifetime of the i-th discrete component; or a sum of Gaussian distribution functions as in the equation

$$I ( t ) = {I}_{o} {\sum }_{i} {α}_{i} {τⅇ}^{t / τ}$$

and

$${α}_{i} ( τ ) = {( \frac{1}{σ \sqrt{2} π} ⅇ )}^{- \frac{1}{2} {( \frac{t - τ}{σ} )}^{2}}$$

where τ is the center value of the lifetime distribution and σ is the standard deviation of the Gaussian, which is related to the full width at half-maximum by 2.354 σ. In the frequency domain, the measured quantities at each frequency ω, are the phase shift (Øω) and demodulation factor (m_{ω}) of the emitted light versus the reference light.

Fractional intensity, amplitude, and lifetime parameters were recovered by a non-linear least squares procedure using the software developed at the Center for Fluorescence Spectroscopy. The measured data were compared with calculated values (Ø_{cω},m_{cω}) and the goodness of fit was characterized by

$${χ}_{R}^{2} = \frac{1}{υ} {\sum }_{ω} {( \frac{{φ}_{ω} - {φ}_{c ω}}{δ φ} )}^{2} + \frac{1}{υ} {\sum }_{ω} {( \frac{{m}_{ω} - {m}_{c ω}}{δ m} )}^{2} ,$$

where υ is the number of degrees of freedom and δØ and δm are the uncertainties in the measured phase and modulation values, respectively. The sum extends over all frequencies (ω).

### Example 6

Using Fluorescence Lifetime Spectroscopy to Study Ligand-Induced Conformational Changes in the β₂AR.

The β₂AR was purified and labeled at Cys265 with fluorescein maleimide to generate FM-β₂AR as previously described. Ligand-dependent changes in fluorescence lifetime of FM-β₂AR were examined in an effort to identify the existence of agonist-specific conformational states. Fluorescence lifetime analysis can detect discrete conformational states in a population of molecules, while fluorescence intensity measurements reflect the weighted average of one or more discrete states.

Based on the observed changes in steady-state fluorescence intensity, it was predicted that ligand-induced conformational changes in the receptor would alter the fluorescence lifetime of the fluorophore. Fluorescence lifetime, τ, refers to the average time that a fluorophore which has absorbed a photon remains in the excited state before returning to the ground state. The lifetime of fluorescein (nanoseconds) is much faster than the predicted off-rate of the agonists we examined (μs-ms), and much shorter than the half-life of conformational states of bacteriorhodopsin (μs) (Subramaniam, et al. (2000) Nature 406(6796), 653-7), rhodopsin (ms) (Farahbakhsh, et al. (1993) Science 262(5138), 1416-9; Arnis, et al. (1994) J Biol Chem 269(39), 23879-81) or of ion channels (μs-ms) (Hoshi, et al. (1994) J Gen Physiol 103(2), 249-78). Therefore, lifetime analysis of fluorescein bound to Cys265 is well-suited to capture even short-lived, agonist-induced conformational states.

### Example 7

Antagonist Binding Narrows the Distribution of Fluorescence Lifetimes

Data from fluorescence lifetime experiments on FM-β₂AR bound to different drugs at equilibrium were analyzed in two ways. Traditionally, fluorescence decays are fit to single and multiple discrete exponential functions and the best fit determined by χ² analysis. In this analysis, t he observed fluorescence decay was resolved into one or more exponential components, with each component, i, being described by τᵢ and τᵢ, where τᵢ represents the fractional contribution of τᵢ to the overall decay. The best fit to single or multiple components was determined by χ² analysis. If different agonists induce a single active state, then the fluorescence lifetime associated with that state (τ_{R*}) should be the same for different drugs and only the fractional contributions (τ_{DRUG}) should differ. However, if there are agonist-specific conformational states we should observe unique, agonist-specific lifetimes (e.g. τ_{ISO}, τ_{SAL}, and τ_{DOB})

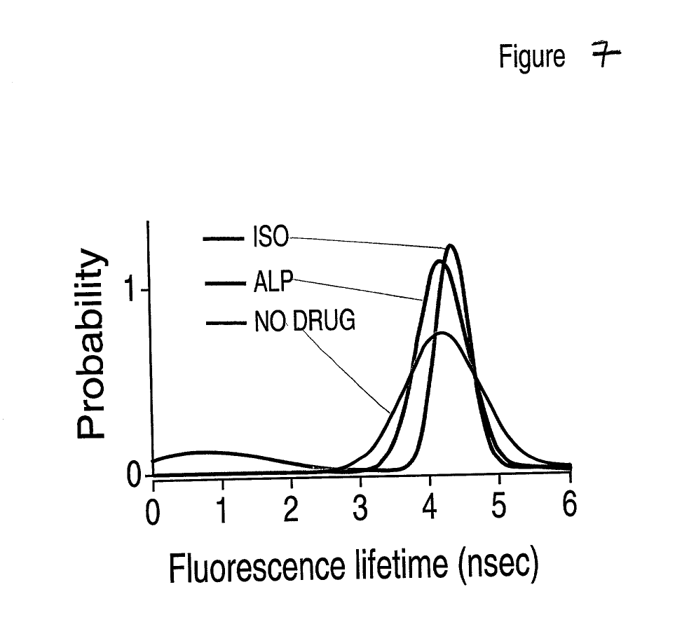

This discrete component analysis assumes that the receptor exists in one or a few rigid protein conformations and does not accurately reflect the dynamic nature of proteins. Proteins that are functionally in a single conformational state actually undergo small conformational fluctuations around a minimum energy state (Frauenfelder, et al. (1991) Science 254(5038), 1598-603) and these small structural perturbations can lead to small changes in the environment around an attached fluorophore. These perturbations are thought to reflect local unfolding reactions within the three dimensional structure of proteins (Freire, E. (2000) Proc Natl Acad Sci U S A 97(22), 11680-2). Such flexibility in protein structure can be modeled using fluorescence lifetime distributions (Gratton, et al. (1989) in Fluorescent Biomolecules: Methodologies and Applications (Jameson, D. M., ed), pp. 17-32, Plenum Press, New York), wherein the width of the distributions reflects the conformational flexibility of the protein (FIG. 7). The mobility of fluorescein relative to the receptor is minimal, as determined by its high measured anisotropy (r=0.30±0.02, n=3), and therefore would be expected to contribute little to the width of the lifetime distribution. Thus, the width of the distribution can be attributed to conformational flexibility in the receptor itself.

Lifetime analysis of unliganded FM-β₂AR reveals a single, flexible state. This is indicated by both the single, broad Gaussian distribution of lifetimes centered around 4.2 ns (FIG. 7, black trace), and the discrete component analysis, where the fluorescence decay rate of FM-β₂AR in the absence of any drug is best fit by a single exponential function (Table 1). Binding of the neutral antagonist ALP to FM-β₂AR does not significantly change the fluorescent lifetime (Table 1), but does narrow the distribution of lifetimes (FIG. 7, red trace), suggesting that ALP stabilizes the receptor and reduces conformational fluctuations. This interpretation is consistent with the results of experiments demonstrating that the β₂AR is more resistant to protease digestion when bound to ALP (Kobilka, B. K. (1990) J Biol Chem 265(13), 7610-8).

TABLE 1
Fluorescent lifetime data for FM-β₂AR in the presence and absence
of drugs fit to discrete exponential functions.

| | τ₁ (nsec) | τ₂ (nsec) | α₂ | χ² |
|---|---|---|---|---|
| NO DRUG | 4.22 ± 0.02 | — | — | 2.9 ± 0.4 |
| ALP | 4.21 ± 0.01 | — | — | 3.1 ± 0.8 |
| ISO | 4.30 ± 0.01 | 0.77 ± 0.05 | 0.19 ± 0.03 | 3.3 ± 1.0 |
| SAL | 4.35 ± 0.02 | 1.45 ± 0.16 | 0.08 ± 0.01 | 2.0 ± 0.2 |
| DOB | 4.36 ± 0.01 | 1.68 ± 0.3 | 0.07 ± 0.01 | 1.8 ± 0.4 |

### Example 8

Agonists and Partial Agonists Induce Distinct Conformations

Unexpectedly, binding of the full agonist ISO promotes conformational heterogeneity. In the presence of saturating concentrations of ISO, FM-β₂AR has two distinguishable fluorescence lifetimes (FIG. 7 and Table 1) representing at least two distinct conformational states. The long lifetime component is only slightly longer than the lifetime observed in the absence of drugs; however, the distribution is narrower than that observed in the presence of the antagonist ALP (FIG. 7, compare green and red traces). In contrast, the distribution of the short lifetime component observed in the presence of ISO is relatively broad, suggesting that there is considerable flexibility around Cys265 in this agonist-induced conformation.

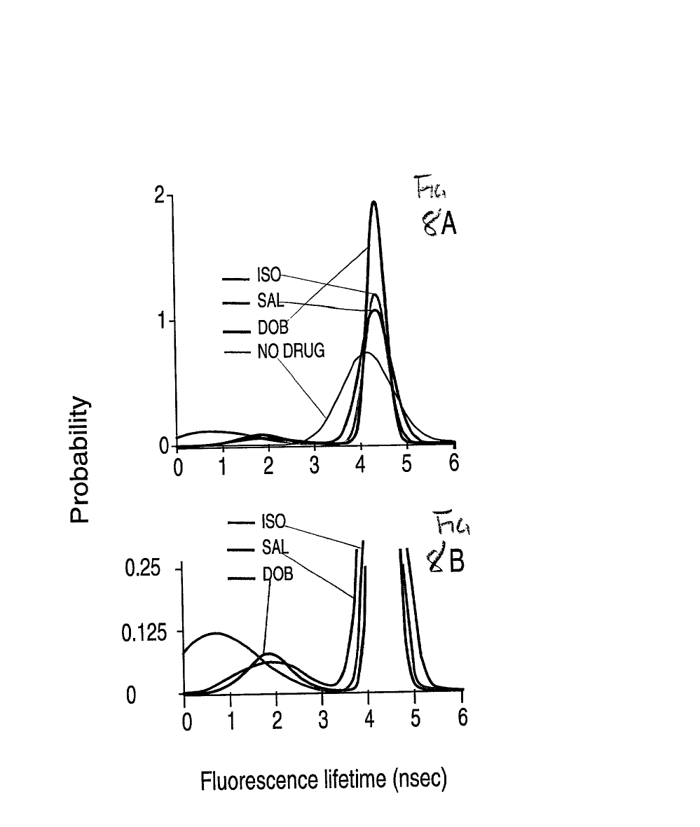

The effect of the partial agonists salbutamol (SAL) and dobutamine (DOB) on the fluorescence lifetime of FM-β₂AR was next examined. Similar to ISO, we observed two lifetimes when the receptor was bound to saturating concentrations of SAL and DOB (Table 1 and FIGS. 8A-8B). The long lifetime component found in the presence of these two partial agonists is indistinguishable from that observed in the ISO-bound receptor; however, the short lifetime component found in both the SAL- and DOB-bound receptor is statistically different from that for the ISO-bound receptor. A strong correlation was observed between a reduction in fluorescence intensity of FM bound to Cys265 and drug efficacy, and shortening of the average fluorescence lifetime is associated with a reduction in fluorescence intensity. Therefore, the short lifetime, found only in the presence of agonists, likely represents the G protein activating conformation of FM-β₂AR.

The different short lifetimes for the full agonist (ISO) and the partial agonists (SAL and DOB) indicate different molecular environments around the fluorophore and therefore represent different, agonist-specific active states. The narrowing and rightward shift of the long lifetime component following binding of both agonists and partial agonists indicate that this lifetime also reflects an agonist-bound state, but most likely represents a more abundant intermediate state that would not be expected to alter greatly the intensity of FM bound to Cys265. It is possible that the number of conformations that we observe in these experiments represent only a few of the possible conformations that can be stabilized by drugs. Moreover, while the overlapping short lifetime distributions of SAL and DOB (FIG. 8B and Table 1) suggest that they induce similar conformations, it is possible that a conformationally sensitive probe positioned elsewhere on the receptor could distinguish between DOB- and SAL-bound receptors states.

### Example 9

Models of GPCR Activation

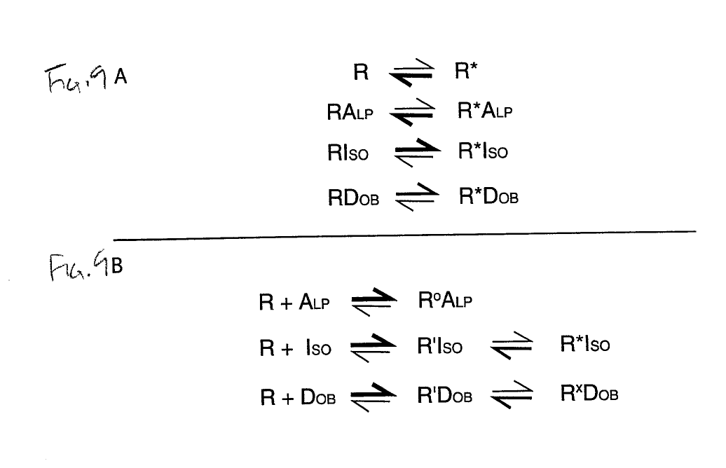

According to the prevailing two-state model of GPCR activation, receptors exist in an equilibrium between a resting (R) state and an active (R*) state which stimulates the G protein (Samama, et al. (1993) J Biol Chem 268(7), 4625-36; 30. Lefkowitz, et al. (1993) Trends Pharmacol Sci 14(8), 303-7; Leff, P. (1995) Trends Pharmacol Sci 16(3), 89-97). Agonists preferentially enrich the R* state, while inverse agonists select for the R state of the receptor. Neutral antagonists possess an equal affinity for both states and function simply as competitors. In this simple model, functional differences between drugs can be explained by their relative affinity for the single active R* state (FIG. 9A). Alternatively, differences in efficacy between drugs have been explained by ligand-specific receptor states (Kenakin, T. (1997) Trends Pharmacol Sci 18(11), 416-7; Tucek, S. (1997) Trends Pharmacol Sci 18(11), 414-6; Strange, P. G. (1999) Biochem Pharmacol 58(7), 1081-8). Our lifetime experiments can best be explained by a model with multiple agonist-specific active states (FIG. 9B).

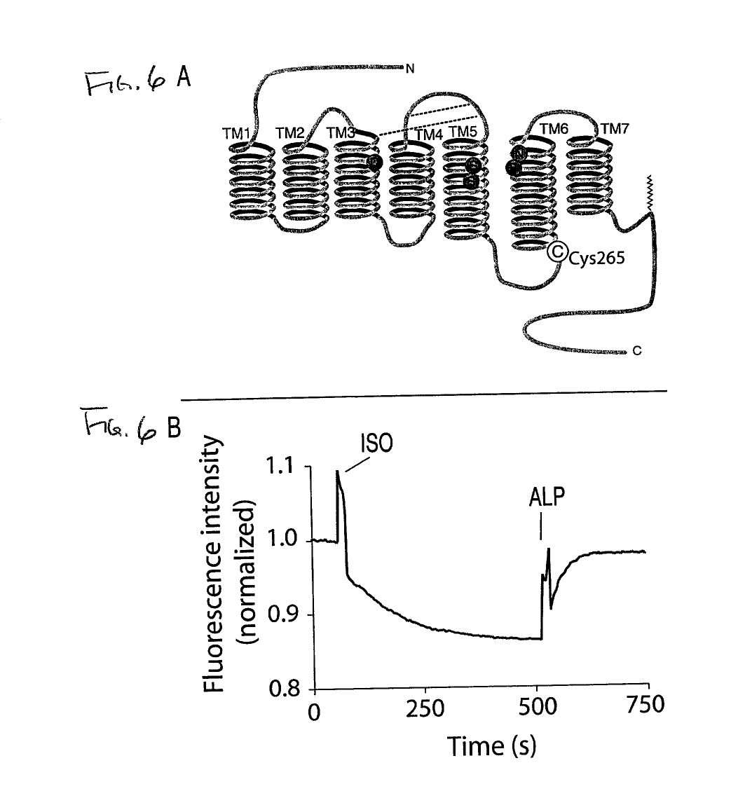

Based on these data, and without being held to theory, the inventors propose a model whereby receptor activation occurs through a sequence of conformational changes. Upon agonist binding, the receptor undergoes a conformational change to an intermediate state (R′) that is associated with a narrowing and rightward shift in the long lifetime distribution. The less abundant active state, represented by the short lifetime, is different for the full agonist ISO (R*) and the partial agonists DOB and SAL (R^{X}). The relatively slow, temperature-dependent rate of change of fluorescence intensity following agonist binding and the rapid rate of reversal by antagonist and FIG. 6B) suggest that transitions from the intermediate state to the active state are relatively rare high energy events. It is likely that in vivo the active conformation is further stabilized by interactions between the receptor and its cognate G protein Gₛ. Thus, one might expect the proportion of receptor in the active state to be greater when the receptor is coupled with Gₛ.

Conclusions

The results described above have implications for drug discovery and efforts to obtain high resolution crystal structures of GPCRs. The results described herein indicate that GPCRs are relatively plastic. The number of conformations that we observed in these experiments may represent only a few of a larger spectrum of possible conformations that could be stabilized by drugs. Thus, it may be possible to identify even more potent agonists or agonists that can alter G protein coupling specificity. Furthermore, these findings indicate that the conformational changes associated with β₂AR activation are similar to those in rhodopsin (Farrens, et al. (1996) Science 274(5288), 768-70) and indicate a shared mechanism of GPCR activation.

The effect of agonists and partial agonists on the fluorescence intensity of FM-β₂AR correlates well with their biological properties. Binding of the full agonist isoproterenol to FM-β₂AR induces a conformational change that leads to a decrease in fluorescence intensity of FM bound to Cys265 by ˜15% (FIG. 6B), while binding of partial agonists results in a smaller change in intensity and binding of antagonists has no effect. Agonist-induced movement of FM bound to Cys265 was characterized by examining the interaction between the fluorescein at Cys265 and fluorescence quenching reagents localized to different molecular environments of the receptor. By site-specific labeling with a single fluorophore on the cytoplasmic extension of TM6 and with a single quencher on the cytoplasmic extension of TM5, evidence was obtained and described herein for movement of these two labeling sites toward each other. This observation and the results of studies using either an aqueous quencher or quenchers that partition into the detergent micelle are most consistent with either a clockwise rotation of TM6 and/or a tilting of the cytoplasmic end of TM6 toward TM5.

These results provide insight into the nature of the structural changes that occur upon agonist binding. Using conventional spectroscopy, no change in the fluorescence intensity from FMβ₂AR upon antagonist binding. This could indicate that antagonists do not alter receptor structure or that the structural changes are not detectable by FM bound to Cys265.

Of greater interest is the structural basis of partial agonism. Partial agonists induce a smaller change in intensity of FM-β₂AR than do full agonists. Without being held to theory, two models could explain this observation. If it is assumed that the receptor exists in two functional conformational states, inactive or active, then a partial agonist may simply induce a smaller fraction of receptors to undergo the transition to the active state than does the full agonist. Alternatively, partial agonists may induce a conformation distinct from that induced by full agonists. Conventional fluorescence spectroscopy, which represents an average intensity over a population of fluorescent molecules, does not distinguish between these two models. Fluorescence lifetime spectroscopy studies indicated that partial agonists and agonists induce distinct conformations. Moreover, structural effects of antagonist binding were observed that could not be detected by conventional spectroscopy. These results help elucidate the structural mechanisms which underlie ligand efficacy, and further aid rational drug design.

### Example 10

Protease Digestion of FM-β2AR is used to Detect Ligand-Specific Conformational States.

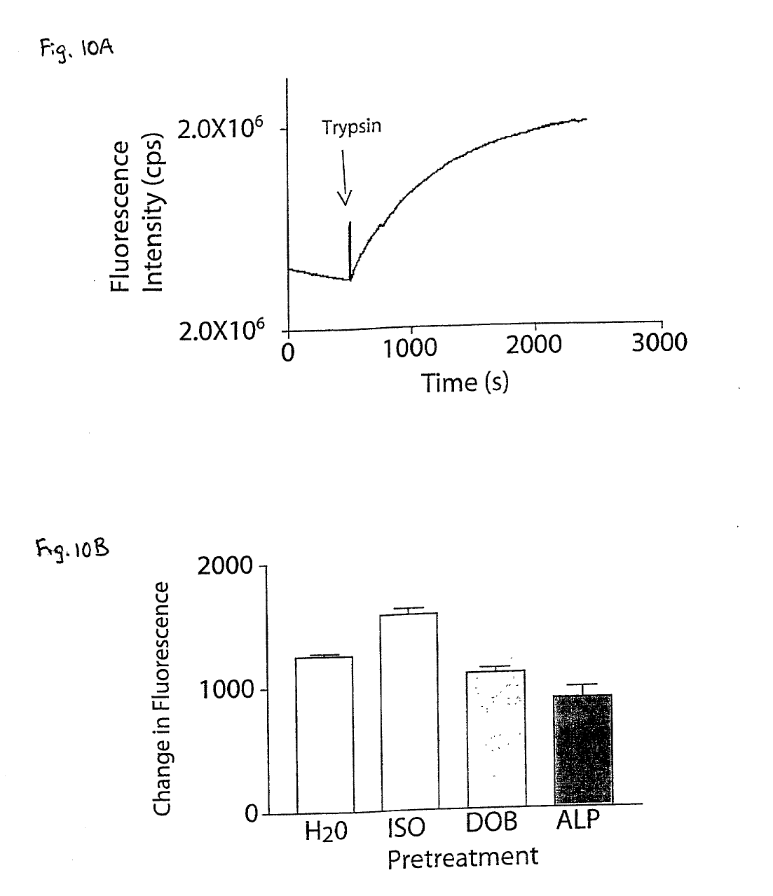

Treatment of FM-β2 AR with the protease trypsin was found to cause an increase in the fluorescence intensity from FM-β2AR over time, most likely due to its action at one or more basic amino acids in the third loop adjacent to Cys265 (See FIG. 10A). The initial rate of digestion, as reflected in the rate of fluorescence increase, after pretreatment with ISO was greater than the rate in the absence of drugs. In contrast, DOB or ALP pretreatment reduced the rate of tryptic digestion relative to treatment with water (see FIG. 10B). Thus, the rate of cleavage is faster when the GPCR is in the presence of agonists, and slower when the GPCR is in the presence of antagonists and partial agonists.

### Example 11

Modified β2-AR having Introduced Protease Cleavage Site(s) as Conformationally Sensitive Detectable Probe

In one embodiment, the conformationally sensitive probe is a protease cleavage site introduced into the GPCR. This can be accomplished by, for example, introducing a protease cleavage site into the second or third intracellular loop of the GPCR. This is exemplified in FIG. 12, which shows the amino acid sequence of the native human β₂-adrenergic receptor and modifications that can be made within the second intracellular loop or within the third intracellular loop to insert a protease cleavage site. The protease cleavage site in this example is for the protease of the tobacco etch virus (TEV), which recognizes and cleaves at the amino acid sequence ENLYFQG (SEQ ID NO: 2) between the glutamine and glycine residues.

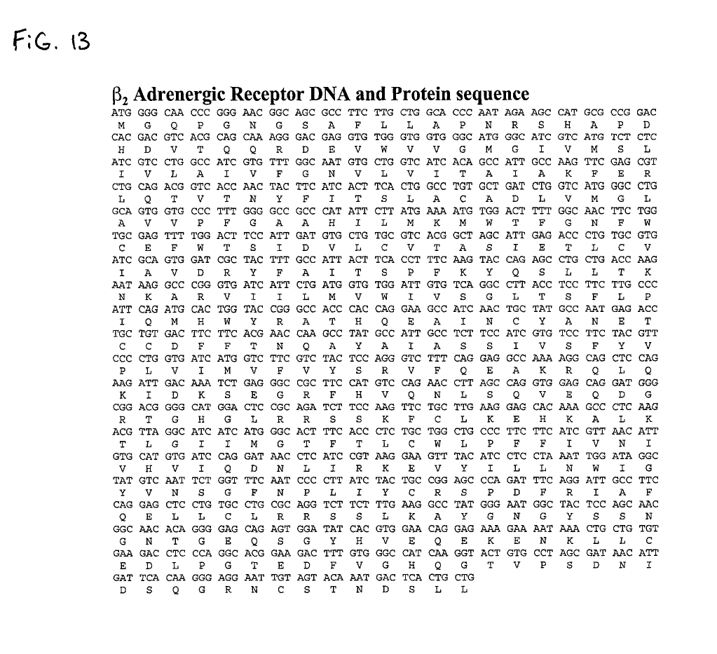

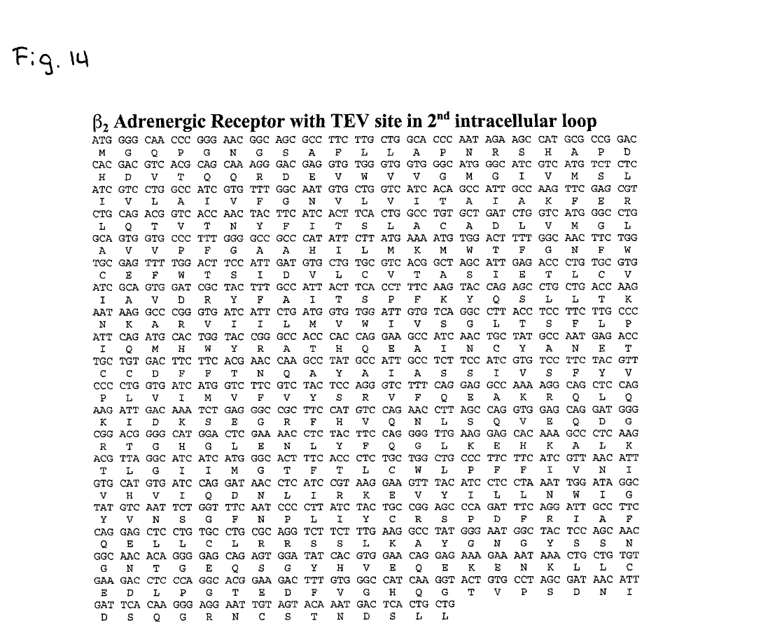

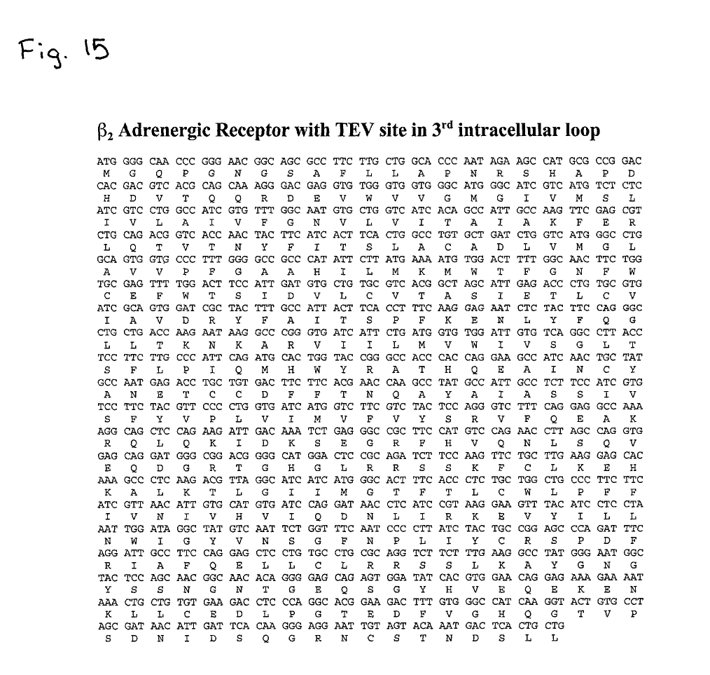

Introduction of the TEV protease cleavage site can be accomplished according to methods well known in the art. The nucleotide and amino acid sequence of native β2-AR are provided in FIG. 13. This sequence is modified to have the amino acid residues in either the second intracellular loop or the third intracellular loop as indicated in FIG. 12. A modified β2-AR having a TEV protease cleavage site in the second intracellular loop can be constructed by modifying the corresponding coding sequence as illustrated in FIG. 14. Similarly, a modified β2-AR having a TEV protease cleavage site in the third intracellular loop can be constructed by modifying the corresponding coding sequence as illustrated in FIG. 15.

### Example 12

Modified μ Opioid Receptor having Introduced Protease Cleavage Site(s) as Conformationally Sensitive Detectable Probe

The μ opioid receptor is another example of a GPCR that can be modified to contain a protease cleavage site as a conformationally sensitive probe. The modified μ opioid receptor can be generated by, for example, introducing a protease cleavage site into the second or third intracellular loop of the GPCR. FIG. 16 is a schematic showing the amino acid sequence of human μ-opioid receptor and modifications that can be made within the second intracellular loop or within the third intracellular loop to insert a protease cleavage site (exemplified by tobacco etch virus (TEV)) that can serve as a conformationally sensitive probe for ligand binding.

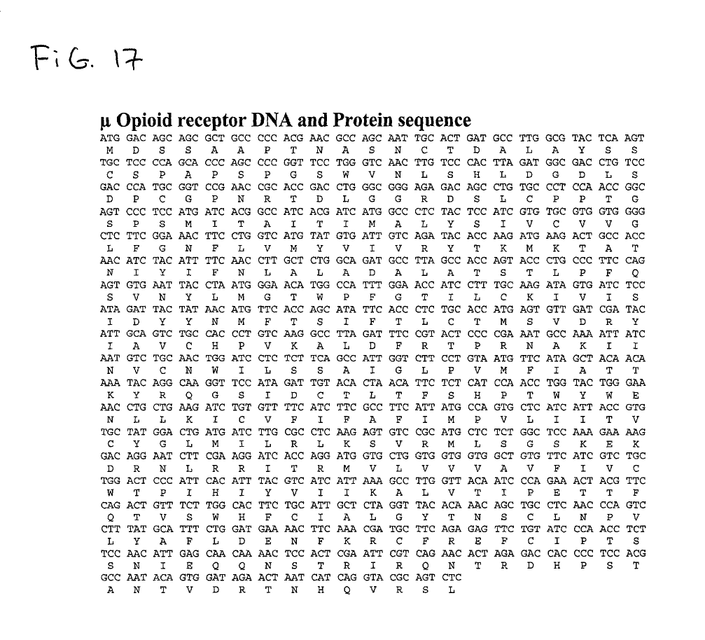

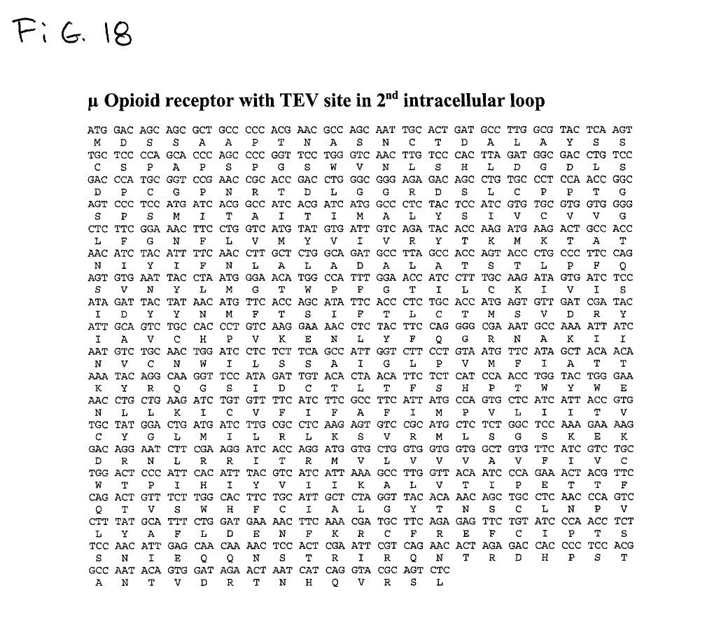

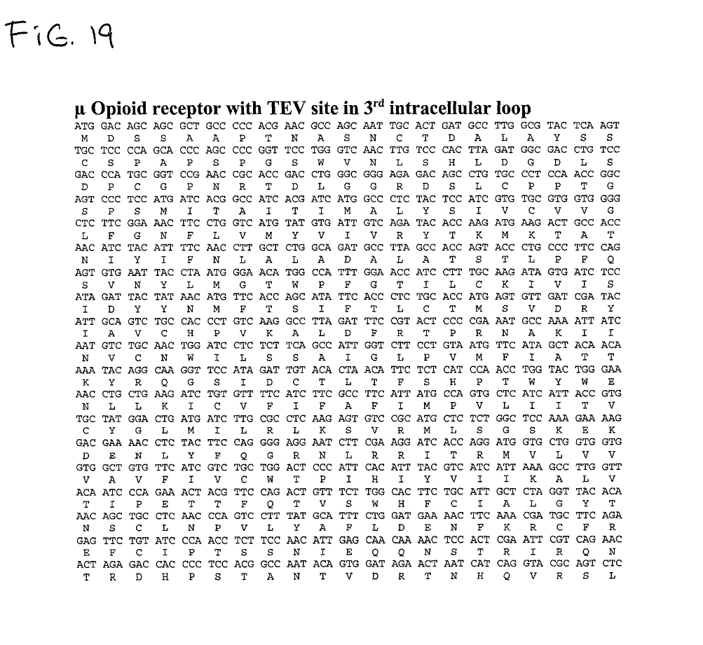

Introduction of the TEV protease cleavage site can be accomplished according to methods well known in the art. The nucleotide and amino acid sequence of native [NOTE: Human?] opioid receptor are provided in FIG. 17. This sequence is modified to have the amino acid residues in either the second intracellular loop or the third intracellular loop as indicated in FIG. 16. A modified μ opioid receptor a TEV protease cleavage site in the second intracellular loop can be constructed by modifying the corresponding coding sequence as illustrated in FIG. 18. Similarly, a modified pt opioid receptor having a TEV protease cleavage site in the third intracellular loop can be constructed by modifying the corresponding coding sequence as illustrated in FIG. 19.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

We claim:

1. A method for identifying an agent having activity agonist activity for a G protein-coupled receptor (GPCR), the method comprising:
contacting a G protein-coupled receptor (GPCR) with a candidate agent, the GPCR having a conformationally sensitive detectable probe positioned on or within a conformationally sensitive third intracellular loop of the GPCR; and
detecting a detectable signal of the conformationally sensitive detectable probe;
wherein detection of a change in the detectable signal in the present of the candidate agent indicates the candidate agent has agonist binding activity for the GPCR.

2. The method of claim 1, wherein the conformationally sensitive intracellular loop is a third intracellular loop of the GPCR and wherein the conformationally sensitive detectable probe is a detectable label attached to one or more amino acid residues within the third intracellular loop of the GPCR so that a conformational change in the GPCR due to agonist activity of the candidate agent causes a change in the detectable signal of the detectable label.

3. The method of claim 2, wherein the detectable label is a fluorescent probe.

4. The method of claim 2, wherein the detectable label is attached to an amino acid residue corresponding to amino acid residue at position 265 in a β2-adrenergic receptor.

5. The method of claim 1, wherein the conformationally sensitive detectable probe is a protease cleavage site. within the GPCR so that a conformational change in the GPCR changes the accessibility of the protease cleavage site to protease cleavage, and the detectable signal is a protease cleavage product.

6. The method of claim 5, wherein the protease cleavage product is an N-terminal fragment of the GPCR.

7. The method of claim 5, wherein the protease cleavage product is an C-terminal fragment of the GPCR.

8. The method of claim 4, wherein the detectable probe comprises two protease cleavage sties within the third intracellular domain of the GPCR, the cleavage sites flanking an epitope tag, wherein a conformational change due to agonist activity changes the accessibility of the protease cleavage site to protease cleavage, and the detectable signal is a polypeptide of the epitope tag released by protease cleavage of the two cleavage sites.

9. The method of claim 1, wherein the GPCR is immobilized by attachment to a support.

10. The method of claim 9, wherein the GPCR is attached to the support by binding of an N-terminal portion to the support.

11. The method of claim 9, wherein the GPCR is attached to the support by binding of an C-terminal portion to the support.

12. The method of claim 1, wherein the GPCR is in a membrane.

13. The method of claim 5, wherein the GPCR is expressed in a eukaryotic host cell.

14. An apparatus for detecting a ligand having agonist activity for a G protein-coupled receptor, the apparatus comprising:
a G protein-coupled receptor (GPCR) with a candidate agent, the GPCR having a conformationally sensitive detectable probe positioned on or within a third intracellular loop of the GPCR; and
a immobilization phase in which the GPCR is positioned.

15. The apparatus of claim 14, wherein the conformationally sensitive detectable probe is a detectable label attached to one or more amino acid residues within the third intracellular loop of the GPCR so that a conformational change in the GPCR due to agonist activity of the candidate agent causes a change in the detectable signal of the detectable label.

16. The apparatus of claim 15, wherein the detectable label is a fluorescent probe.

17. The apparatus of claim 15, wherein the detectable label is attached to an amino acid residue corresponding to amino acid residue at position 265 in a β2-adrenergic receptor.

18. The apparatus of claim 14, wherein the conformationally sensitive detectable probe is a protease cleavage site. within the GPCR so that a conformational change in the GPCR changes the accessibility of the protease cleavage site to protease cleavage, and the detectable signal is a protease cleavage product.

19. The apparatus of claim 14, wherein the detectable probe comprises two protease cleavage sties within the third intracellular domain of the GPCR, the cleavage sites flanking an epitope tag, wherein a conformational change due to agonist activity renders the cleavage sites accessible to protease cleavage, and the detectable signal is a polypeptide of the epitope tag released by protease cleavage of the two cleavage sites.
